(19) United States
(12) Patent Application Publication
(10) Pub. No.: US 2023/0077811 A1
(43) Pub. Date: Mar. 16, 2023

(54) ACTIVATION OF NEUROPEPTIDE RECEPTORS ON PLASMACYTOID DENDRITIC CELLS TO TREAT OR PREVENT OCULAR DISEASES ASSOCIATED WITH NEOVASCULARIZATION AND INFLAMMATION

(71) Applicants: Tufts Medical Center, Inc., Boston, MA (US); Trustees of Tufts College, Medford, MA (US)

(72) Inventors: Pedram HAMRAH, Wellesley, MA (US); Abdo ABOU-SLAYBI, Worcester, MA (US)

(21) Appl. No.: 17/798,344

(22) PCT Filed: Feb. 11, 2021
(86) PCT No.: PCT/US2021/017628

§ 371 Date: Aug. 9, 2022

Related U.S. Application Data
(60) Provisional application No. 62/975,059, filed on Feb. 11, 2020.

(51) Int. Cl. A61K 31/454; A61K 38/33; A61K 9/00; A61P 27/02

(52) U.S. Cl. CPC A61K 31/454; A61K 38/33; A61K 9/0048; A61K 9/0019; A61P 27/02

(57) ABSTRACT

The invention relates to methods and compositions for use in the treatment and prevention of ocular diseases or conditions associated with neovascularization and/or inflammation.

### STATEMENT AS TO FEDERALLY FUNDED RESEARCH

This invention was made with government support under grant numbers EY026963, EY029602, and EY022695 awarded by the National Institutes of Health. The government has certain rights in the invention.

### FIELD OF THE INVENTION

The invention relates to methods and compositions for use in the treatment and prevention of diseases and conditions associated with neovascularization and/or inflammation, such as diseases or conditions of the eye.

### BACKGROUND

Pathological angiogenesis, in part, is caused by a disruption of the balance between angiogenic factors such as vascular endothelial growth factor 1 (VEGF1) and basic fibroblast growth factor 2 (bFGF2), and angiostatic factors such as endostatin (ES) (Folkman, Nat. Med. 1:27-31, 1995; O'Reilly et al., Cell 88:277-285, 1997; Folkman, N. Eng. J. Med. 285:1182-1186, 1971; Lai et al., J. Biomed. Sci. 14:313-322, 2007; Ellenberg et al., Prog. Retin. Eye Res. 29:208-248, 2010), platelet factor 4 (PF4)(Sharpe et al., J. Natl. Cancer Inst. 82:848-853, 1990; Maione et al., Science 247:77-79, 1990; Kolber et al., J. Natl. Cancer Inst. 87:304-309, 1995), thrombospondin 1 (TSP-1) (Lawler, Curr. Opin. Cell Biol. 12:634-640, 2000; Lawler et al., Cold Spring Harb. Perspect. Med. 2:a006627, 2012; Lawler, J. Cell Mol. Med. 6:1-12, 2002; Armstrong et al., Matrix Biol. 22:63-71, 2003; Cursiefen et al., Invest. Ophthalmol. Vis. Sci. 45:1117-1124, 2004; Sekiyama et al., Invest. Ophthalmol. Vis. Sci. 47:1352-1358, 2006), and tissue inhibitor of matrix metalloprotease three (Timp3)(Qi et al., Nat. Med. 9:407-415, 2003; Lee et al., Mol. Vis. 15:2480-2487, 2009). This disruption and induction of angiogenesis is a hallmark and cause of morbidity and mortality in a range of diseases including cancer, diabetes, macular degeneration, and corneal vascularization (CNV). Understanding the complex regulation of angiogenesis may lead to novel therapeutic interventions in these diseases. Regulation of angiogenesis in vivo and in particular the involvement of the nervous and immune systems is an active area of investigation. Separately, the role of both the nervous system and the immune system have been examined on mediating angiogenesis. Yet, neuronal regulation of immune cell mediated angiogenesis and the role of particular immune cells, such as plasmacytoid dendritic cells (pDCs), remains unknown.

Angiogenesis is the development of new vessels from an existing vasculature. A classic model system to examine in vivo induction of angiogenesis is CNV (Folkman, N. Eng. J. Med. 285:1182-1186, 1971; Gimbrone et al., J. Natl. Cancer Inst. 52:413-427, 1974). During CNV, vessels along the periphery extend new sprouts into the cornea from the vascular limbus (Chang et al., Curr. Opin. Ophthalmol. 12:242-249, 2001). CNV can lead to the loss of corneal transparency, decreased visual acuity, rejection of corneal transplants (Lee et al., Surv. Ophthalmol. 43:245-269, 1998), and possibly blindness (Chang et al., Curr. Opin. Ophthalmol. 12:242-249, 2001). The cornea is endowed with resident bone marrow derived leukocytes such as corneal resident Langerhans cells (LCs), mature (CD45+, major histocompatibility complex II (MHC-II+), CD80+,CD86+) and immature (CD45+, MHC-II+, CD80+, CD86+, CD11c+) conventional dendritic cells (cDCs), and (CD45+, CD11b+, CD11c−, F4/80+, Iba-1+) macrophages (Hamrah et al., Arch. Ophthalmol. 121:1132-1140, 2003; Hamrah et al., J. Leukoc. Biol. 74:172-178, 2003; Hamrah et al., Invest. Ophthalmol. Vis. Sci. 44:581-589, 2003; Hamrah et al., Invest. Ophthalmol. Vis. Sci. 44:581-589, 2003). Inflammation of the cornea results in immune recruitment and altered corneal leukocyte populations (Hamrah et al., Arch. Ophthalmol. 121:1132-1140, 2003; Hamrah et al., Antigen-Presenting Cells in the Eye and Ocular Surface 120-127, 2010). Recent studies have also identified a novel set of resident corneal pDCs (murine: CD45+, plasmacytoid dendritic cell antigen 1 (PDCA-1+), CD11c-low, sialic acid binding Ig-like lectin H (Siglec-H+), and the B220 isoform of CD45R (B220+); human: CD11c-low, CD45+, BDCA2+, BDCA4+) (Hamrah et al., Antigen-Presenting Cells in the Eye and Ocular Surface 120-127, 2010; Sosnova et al., Stem Cells 23:507-515, 2005; Forrester, Immunol. Rev. 234:282-304, 2010). The functions and roles of pDCs in the cornea have yet to be fully characterized. In addition to resident leukocytes, the cornea contains approximately 7,000 epithelial layer free nerve endings per square millimeter resulting in the cornea as the most densely innervated tissue in the body (Cruzat et al., Ocul. Surf. 15:15-47, 2017; Millodot, Ophthalmic Physiol. Opt. 4:305-318, 1984; Muller et al., Exp. Eye Res. 76:521-542, 2003). The cornea is innervated by neurons derived from the ciliary nerves of the ophthalmic branch of the trigeminal nerves. Despite high levels of innervation and the presence of corneal leukocytes, the role of corneal nerves directly or indirectly modulating angiogenesis through leukocytes remains to be elucidated.

Leukocytes such as macrophages (Casazza et al., Cancer Cell 24:695-709, 2013; Eslani et al., Stem Cells 36:775-784, 2018; Narimatsu et al., Sci. Rep. 9:2984, 2019; Kiesewetter et al., Sci. Rep. 9:308, 2019; Seyed-Razavi et al., Invest. Ophthalmol. Vis. Sci. 55:1313-1320, 2014), neutrophils (Christoffersson et al., Blood 120:4653-4662, 2012; Gong et al., Cell Tissue Res. 339:437-448, 2010; Tazzyman et al., Int. J. Exp. Pathol. 90:222-231, 2009), and cDCs23 (Hamrah et al., Am. J. Pathol. 163:57-68, 2003; David Dong et al., Curr. Pharm. Des. 15:365-379, 2009; Sozzani et al., Trends Immunol. 28:385-392, 2007) have been extensively implicated in stimulation or modulation of angiogenesis (Kreuger et al., Nat. Rev. Drug Disc. 15:125-142, 2015). Yet, the role of pDCs remains largely unknown. While neuronal regulation of leukocytes has been extensively examined and reviewed (Norris et al., J. Exp. Med. 216:60-70, 2019; Dantzer, Physiol. Rev. 98:477-504, 2018; Marin et al., Learn. Mem. 20:601-606, 2013; Benarroch, Neurology 92:377-385, 2019; Tian et al., Trends Immunol. 30:91-99, 2009) the direct mechanism by which neurons and leukocytes interact through neuropeptides remains unknown (Souza-Moreira et al., Neuroendocrinology 94:89-100, 2011; Ganea, Brain. Behay. Immun. 22:33-34, 2008). Recent studies have begun to shed light on the role of neuropeptides in neuroimmune crosstalk such as the melanocortin system on lymphocytes (Lisak et al., Brain Sciences 7, 2017), calcitonin gene-related peptide (CGRP) on modulating innate lymphoid cells type 2 (ILC2)(Nagashima et al., Immunity 51(4):682-695, 2019), or vasoactive intestinal polypeptide (VIP) enhancing pDC mediated T cell activation (Li et al., Blood 126:3438-3438, 2015). Few, if any, studies have examined the crosstalk between neurons, leukocytes, and vessels using the cornea as a model system. The avascularity of the cornea, coupled with the presence of resident leukocytes, and the high innervation of the cornea, allow for an optimal environment to examine the potential of neuronal modulation of avascularity through corneal leukocytes according to the present disclosure.

There is a need for approaches to prevent and treat diseases and conditions associated with neovascularization and/or inflammation, such as, e.g., diseases or conditions of the eye.

### SUMMARY OF THE INVENTION

The present disclosure provides methods and compositions for use in preventing or treating an ocular disease or condition associated with neovascularization and/or inflammation in a subject (e.g., a human subject). The methods include administering a neuropeptide receptor agonist to the subject (such as by way of administration to the eye or by way of systemic (e.g., intravenous) administration). In various examples, the subject has or is at risk of developing a disease or condition associated with neovascularization and/or inflammation of various tissues of the eye, such as, e.g., neovascularization and/or inflammation of the cornea, retina, or choroid.

In a first aspect, the invention provides a method of treating or preventing a disease or condition characterized by neovascularization and/or inflammation in a subject, the method including activating a neuropeptide receptor on plasmacytoid dendritic cells (pDCs) in the subject (e.g., pDCs of the eye).

In some embodiments, the disease or condition is characterized by neovascularization and/or inflammation is an ocular disease or condition.

In some embodiments, the neovascularization and/or inflammation is corneal neovascularization and/or inflammation. In some embodiments, the subject has or is at risk of developing a corneal infection, inflammation, autoimmune disease, limbal stem cell deficiency, neoplasia, uveitis, keratitis, corneal ulcers, glaucoma, rosacea, lupus, dry eye disease, or ocular damage due to trauma, corneal graft rejection, surgery, or contact lens wear. In some embodiments, the disease or condition is episcleritis, scleritis, uveitis, or retinal vasculitis.

In some embodiments, the neovascularization and/or inflammation is retinal neovascularization and/or inflammation. In some embodiments, the subject has or is at risk of developing ischemic retinopathy, diabetic retinopathy, retinopathy of prematurity, retinal vein occlusion, ocular ischemic syndrome, sickle cell disease, Eales' disease, or macular degeneration.

In some embodiments, the neovascularization and/or inflammation is choroidal neovascularization and/or inflammation. In some embodiments, the subject has or is at risk of developing inflammatory neovascularization with uveitis, macular degeneration, ocular trauma, sickle cell disease, pseudoxanthoma elasticum, angioid streaks, optic disc drusen, myopia, malignant myopic degeneration, or histoplasmosis.

In some embodiments, activating a neuropeptide receptor on pDCs in the subject includes administering a neuropeptide receptor agonist to the subject. In some embodiments, the neuropeptide receptor is a melanocortin (MC) receptor, a somatostatin (SST) receptor, or an opioid receptor. In some embodiments, the MC receptor is an MC4 receptor. In some embodiments, the MC receptor is an MC1, MC2, MC3, or MC5 receptor. In some embodiments, the SST receptor is an SST1, SST2, SST3, SST4, or SST5 receptor. In some embodiments, the opioid receptor is a delta (δ) opioid receptor, kappa (κ) opioid receptor, or mu (μ) opioid receptor. In some embodiments, the neuropeptide receptor agonist is ((3R)—N-[(2R)-3-(4-chlorophenyl)-1-[4-cyclohexyl-4-(1,2,4-triazol-1-ylmethyl)piperidin-1-yl]-1-oxopropan-2-yl]-1,2,3,4-tetrahydroisoquinoline-3-carboxamide (THIQ), PF-00446687, PL-6983, or any one of the neuropeptide receptor agonists recited in Tables 1-3.

In some embodiments, activating the neuropeptide receptor on pDCs in the subject increases expression of one or more angiostatic neuropeptides, increases phosphorylation of protein kinase Co/0 (PKCδ/θ), and/or increases nuclear localization of nuclear factor kappa B (NF-κB) in the pDCs. In some embodiments, the one or more angiostatic neuropeptides are selected from the group consisting of endostatin (ES), platelet factor 4 (PF4), thrombospondin 1 (TSP-1), and tissue inhibitor of matrix metalloprotease three (TIMP3).

In some embodiments, the neuropeptide receptor agonist is administered to the eye of the subject. In some embodiments, administration to the eye includes administration by way of intravitreal injection, sub-retinal injection, sub-conjunctival injection, intracorneal injection, eye drops, ophthalmic pellets, drug-eluting contact lenses, ophthalmic plugs, ophthalmic depot, or intraocular device. In some embodiments, the neuropeptide receptor agonist is administered to the subject by way of systemic administration. In some embodiments, the systemic administration includes intravenous injection or infusion.

In some embodiments, the subject is a human.

In another aspect, the disclosure provides a pharmaceutical composition including a neuropeptide receptor agonist and a pharmaceutically acceptable carrier or diluent (e.g., an ophthalmic carrier or diluent).

In another aspect, the disclosure provides a kit including the pharmaceutical composition of the foregoing aspect a topical anesthetic eye drop, and a package insert. In some embodiments, the package insert instructs a user of the kit to perform the method of the disclosure.

In other aspects, the disclosure provides compositions for use in carrying out the methods described herein, use of the compositions for the methods, and use of the compositions for the preparation of medicaments for these uses.

### BRIEF DESCRIPTION OF THE DRAWINGS

FIGS. 1A-1F are a series of images showing expression of angiostatic factors in murine and human plasmacytoid dendritic cells (pDCs). (FIG. 1A) Quantitative real-time polymerase chain reaction (qRT-PCR) of corneal pDCs (PDCA1+, B220+, CD45+), cDCs (CD45+, CD11c+), macrophages (IBA-1+, F4/80+) and splenic pDCs for endostatin (ES), platelet factor 4 (PF4), thrombospondin 1 (TSP-1), and tissue inhibitor of metalloprotease 3 (TIMP3). (FIG. 1B) Flow cytometry gating strategy for splenic pDCs stained for live dead, PDCA1+, B220+, CD45+, SiglecH+, and angiostatic proteins. (FIG. 10) Flow cytometry gating strategy for corneal pDCs stained for live dead, PDCA1+, B220+, CD45+, SiglecH+, and angiostatic proteins. (FIG. 1D) Quantification of murine pDCs for ES, TSP-1, PF4, TIMP3 by flow cytometry. (FIG. 1E) Flow cytometry gating strategy for human corneal pDCs. (FIG. 1F) Phenotypic flow cytometry analysis of human corneal pDCs for ES, TSP-1, PF4, or TIMP3.

FIGS. 2A-2M are a series of images showing neuronal regulation of expression of pDC angiostatic proteins by corneal nerves through the melanocortin 4 (MC4) receptor. (FIG. 2A) Immunofluorescence imaging of corneal pDCs associated with subbasal nerves stained for B3 tubulin. (FIG. 2B) Representative bright-field images of splenic pDCs (i) trigeminal ganglion neurons (TG) (ii) or co-culture of pDCs with TG neurons (iii). (FIG. 2C) qRT-PCR levels of ES, TSP-1, PF4, and TIMP3 in splenic pDCs, TG neuronal cells, and pDCs co-cultured with TG neurons. (FIG. 2D) qRT-PCR levels of ES, TSP-1, PF4, and TIMP3 in splenic pDCs, TG conditioned media, and pDCs incubated with TG conditioned media. (FIG. 2E) Flow cytometry gating strategy for splenic pDCs (live dead, PDCA1+, B220+, SiglecH+, Ly6C+) after co-culture with TG neurons. (FIG. 2F) Quantification of murine pDCs, TG neurons, and pDCs co-cultured with TG neurons for ES, TSP-1, PF4, TIMP3 by flow cytometry. (FIG. 2G) Flow cytometry gating strategy for corneal pDCs after axotomy of TG (live dead, PDCA1+, B220+, SiglecH+, Ly6C+). (FIG. 2H) Quantification of murine corneal pDCs at baseline, in sham, and after corneal axotomy for ES, TSP-1, PF4, TIMP3 by flow cytometry. (FIG. 2I) qRT-PCR of TG POMC expressed by splenic pDCs (autocrine signaling) or trigeminal ganglion neurons (paracrine signaling) by qRT-PCR. (FIG. 2J) qRT-PCR of melanocortin receptors 1-5 by corneal cDCs, or corneal pDCs. (FIG. 2K) Flow cytometry gating strategy of TG neurons for POMC expression. (FIG. 2L) Phenotypic flow cytometry analysis of human corneal pDCs for MC4 receptor. (FIG. 2M) Quantification of murine pDCs after co-culture with TG neurons, with TG neurons pretreated with control siRNA, or TG neurons pretreated with siRNA against POMC for ES, TSP-1, PF4, TIMP3 by flow cytometry. Statistics: (FIGS. 2C, 2D, 2I, and 2J) Normalized to GAPDH, 2-way ANOVA with Tukey Test, error bars are standard deviation n=3 biological replicates *=p<0.05. (FIGS. 2E, 2F, 2G, 2H, and 2M) Compiled representative data, n=10 pooled murine corneas or n=5 pooled spleens. (FIG. 2L) n=3 human corneas, error bars are standard deviation, t-test to baseline *=p<0.05.

FIGS. 3A-3P are a series of images showing that the MC4 agonist, ((3R)—N-[(2R)-3-(4-chlorophenyl)-1-[4-cyclohexyl-4-(1,2,4-triazol-1-ylmethyl)piperidin-1-yl]-1-oxopropan-2-yl]-1,2,3,4-tetrahydroisoquinoline-3-carboxamide (THIQ), increases pDC ES, TSP-1, PF4, and TIMP3 expression and reduces corneal neovascularization. (FIG. 3A) Flow cytometry gating strategy for splenic pDCs (live dead, CD45+, PDCA1+, B220+, SiglecH+, Ly6C+) after co-culture with TG neurons. (FIG. 3B) Flow cytometry plots of murine pDCs incubated with THIQ (10 ug/mL) for ES, TSP-1, PF4, and TIMP3. (FIG. 3C) Gating strategy of human corneal pDCs incubated with selective MC4 agonist THIQ. (FIG. 3D) Flow cytometry plots of human pDCs incubated with THIQ for ES, TSP-1, PF4, and TIMP3. (FIG. 3E) Representative bright-field image of suture induced corneal neovascularization at day 1 (left panel) and day 14 (right panel). (FIG. 3F) Graphical schematic of corneal suture model. (FIG. 3G) Graphical representation of subconjunctival injection timeline. (FIG. 3H) Immunofluorescence of murine corneas for DAPI (blue), CD31 (red) in saline or THIQ (10 mg/mL) injected corneas. (FIG. 3I) Quantification of CNV after saline or THIQ (10 mg/mL) treatment. (FIG. 3J) Graphical schematic of corneal suture model with or without control siRNA or MC4 siRNA. (FIG. 3K) Graphical representation of subconjunctival injection timeline. (FIG. 3L) Immunofluorescence of murine corneas for DAPI (blue), CD31 (red) in control siRNA, MC4 siRNA, control siRNA with THIQ (1 mg/mL), or MC4 siRNA with THIQ (10 ug/mL). (FIG. 3M) Quantification of CNV after control siRNA or MC4 siRNA with or without THIQ (10 ug/mL) treatment. (FIG. 3N) Graphical schematic of high-risk corneal transplant model. (FIG. 3O) Immunofluorescence of murine corneas for DAPI (blue), CD31 (red) in saline or THIQ (10 mg/mL) injected corneas after corneal transplant. (FIG. 3P) Quantification of CNV in high-risk corneal transplant after saline or THIQ (10 ug/mL) injection. Statistics: (FIGS. 3B and 3D) Compiled representative data, n=10 pooled murine corneas or n=3 human corneas *=p<0.05. (FIGS. 31, 3M, 3P) n=4-10 murine corneas, error bars are standard deviation, t-test to baseline *=p<0.05.

FIGS. 4A-4E are a series of images showing increase of expression of angiostatic proteins by pDCs following stimulation of pDC MC4 receptor. (FIG. 4A) Immunoblots of sorted murine pDCs treated with THIQ (10 ug/mL) for known PKC isoforms and associated beta actin. (FIG. 4B) Densitometry of PKC −δ/θ phosphorylation, normalized to beta actin. (FIG. 4C) Immunoblots of sorted murine pDCs treated with THIQ (10 ug/mL) for NF-κB signaling and associated beta actin. (FIG. 4D) Immunofluorescence of sorted pDCs treated with THIQ (10 ug/mL) for Rel-B cytoplasmic and nuclear localization. (FIG. 4E) Immunofluorescence of sorted pDCs treated with THIQ (10 ug/mL) for c-Rel cytoplasmic and nuclear localization. Statistics: (FIG. 4A) Compiled representative data (FIG. 4B) n=5 murine spleens sorted for 500,000 pDCs. *=p<0.05, error bars are standard deviation, t-test to baseline *=p<0.05.

FIGS. 5A-5B are a series of images showing pDC expression of neuropeptide receptors SST4, MC4, NPR2, and delta and kappa opioid receptors. (FIG. 5A) Flow cytometry gating strategy for murine pDCs. (FIG. 5B) Flow cytometry plots of murine pDCs showing pDC expression of SST4, MC4, NPR2, and delta and kappa opioid receptors.

FIGS. 6A-6E are a series of images showing pDC expression of angiostatic molecules, ES and TSP1, following treatment with one of several neuropeptide receptor agonists. (FIGS. 6A and 6B) Flow cytometry plot of splenic pDCs treated with a pan opioid receptor agonist, Dynorphin A. (FIG. 6C) Flow cytometry plot of splenic pDCs treated with a K opioid receptor agonist, U50488. (FIGS. 6D and 6E) Flow cytometry plot of splenic pDCs treated with an SST4 receptor agonist, L-803,087.

FIG. 7 is a summary diagram showing a detailed mechanism by which innervation of the cornea by TG nerve fibers from the subbasal nerve plexus results in neuronal release of pro-opiomelanocortin which activates MC4 receptors on pDCs, resulting in increased PKC phosphorylation and nuclear localization of NF-κB, and subsequent upregulation of angiostatic factors by pDCs (e.g., ES, PF4, TSP-1, and TIMP3).

### DEFINITIONS

As used herein, “administration” refers to providing or giving a subject a therapeutic agent (e.g., a neuropeptide receptor agonist disclosed herein), by any effective route. Exemplary routes of administration are described herein and below (e.g., administration to the eye (e.g., intravitreal injection, sub-retinal injection, sub-conjunctival injection, intracorneal injection, eye drops, ophthalmic pellets, drug-eluting contact lenses, ophthalmic plugs, ophthalmic depot, or intraocular device), and parenteral (e.g., intravenous injection or infusion).

As used herein, the term “agonist” refers to an agent (e.g., a small molecule) that increases receptor (e.g., neuropeptide receptor) activity. An agonist may activate a receptor by directly binding to the receptor, by acting as a cofactor, by modulating receptor conformation (e.g., maintaining a receptor in an open or active state). An agonist may increase receptor activity by 10%, 20%, 30%, 40%, 50%, 60%, 70%, 80%, 90%, 95%, 98%, or more (e.g., at least 100% 150%, 200%, 300%, 400%, 500%, or more; or a range between any of the listed percentages). An agonist may induce maximal receptor activation or partial activation depending on the concentration of the agonist and its mechanism of action.

As used herein, the term “choroidal neovascularization” refers to a sight-threatening condition of the eye characterized by the growth of new blood vessels originating from the choroid by breaching the Bruch membrane into the subretinal pigment epithelium or subretinal space. Choroidal neovascularization is commonly associated with subretinal bleeding, collection of subretinal fluids, lipid exudation, detachment of the retinal pigment epithelium, and subretinal fibrosis. Based on its location to the fovea, choroidal neovascularization may be considered extrafoveal (0.2-1.5 mm from the fovea), juxtafoveal (0.001-0.199 mm from the fovea), or subfoveal. Choroidal neovascularization may be treated or prevented according to the methods and compositions disclosed herein.

As used herein, the terms “corneal neovascularization” or “CNV” refer to a sight-threatening condition of the eye characterized by the growth of new blood vessels from the pericorneal plexus into the normally avascular corneal tissue due to ischemic challenge or a pathophysiological condition. CNV may be inherited or acquired. In cases of acquired CNV, common causes may include inflammation, infection, degeneration, and traumatic or iatrogenic conditions. CNV may be prevented or treated according to the methods and compositions disclosed herein.

As used herein, the terms “increasing” and “decreasing” refer to modulating resulting in, respectively, greater or lesser amounts, of function, expression, or activity of a metric relative to a reference. For example, subsequent to administration of a neuropeptide receptor agonist in a method described herein, the amount of a marker of a metric (e.g., neovascularization and/or production of inflammatory cytokines or chemokines) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95%, or 98% or more (e.g., at least 100% 150%, 200%, 300%, 400%, 500%, or more; or a range between any combination of the listed percentages) relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “inflammation” refers to a complex biological response of the immune system to potentially harmful agents such as, e.g., damaged cells or irritants. Inflammation acts to remove the precipitating cause of cell injury, clear out cellular debris, and initiate tissue repair. Inflammation may be characterized by sensations of heat, pain, redness, swelling, and loss of function of the inflamed tissue. Inflammation can be acute or chronic, depending on the duration of the response and the recovery of the immune system to homeostatic equilibrium. Inflammation may be mediated by secreted factors, such as cytokines or chemokines, produced by immune cells. Inflammation may be a symptom associated with one or more diseases or conditions, such as, e.g., diseases or conditions of the eye disclosed herein.

As used herein, the term “neovascularization” refers to a biological process by which new blood vessels are formed, typically in the form of functional microvascular networks capable of perfusion by red blood cells and serving as collateral circulation in response to low local perfusion or ischemia. Neovascularization may occur in various tissues of the body, including tissues of the eye (e.g., cornea, retina, or choroid). Neovascularization can be modulated by agents disclosed herein, such as, e.g., neuropeptide receptor agonists.

As used herein, the term “neuropeptide receptor” refers to a type of peptide receptor capable of binding one or more neuropeptides to elicit a cellular response. Non-limiting examples of neuropeptide receptors include melanocortin receptors, somatostatin receptors, and opioid receptors.

As used herein, the term “ocular” refers to the eye, including any and all of its cells including muscles, nerves, blood vessels, tear ducts, and membranes, as well as structures that are connected with the eye and its physiological functions. The terms ocular and eye are used interchangeably throughout this disclosure. Non-limiting examples of cell types within the eye include cells located in the ganglion cell layer, the inner plexiform layer inner, the inner nuclear layer, the outer plexiform layer, outer nuclear layer, outer segments (OS) of rods and cones, the retinal pigmented epithelium, the inner segments of rods and cones, the epithelium of the conjunctiva, the iris, the ciliary body, the corneum, and epithelium of ocular sebaceous glands.

As used herein, the term “retinal neovascularization” refers to a sight-threatening condition of the eye characterized by the growth of new blood vessels on the retinal surface, commonly in response to ischemic challenge. Retinal vascularization may threaten vision due to reduced integrity of the new vessels, which results in spontaneous bleedings that cause retinal hemorrhages and attract fibroglial elements that induce vitreous contraction. This may result in further retinal bleeding and detachment. Retinal neovascularization may be treated or prevented using the methods and compositions disclosed herein.

As used herein, the terms “subject” and “patient” refer to an animal (e.g., a mammal, such as a human). A subject to be treated according to the methods described herein may be one who has been diagnosed with a disease or condition associated with neovascularization and/or inflammation, such as a disease or condition of the eye, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

### DETAILED DESCRIPTION

The present disclosure provides methods and compositions for use in treating or preventing ocular diseases and conditions associated with increased neovascularization and/or inflammation in a subject in need thereof by activating a neuropeptide receptor expressed on plasmacytoid dendritic cells (pDCs) of the subject (e.g., pDCs of the eye). The methods and compositions of the disclosure can be used to prevent or treat ocular diseases or conditions characterized by neovascularization, such as, e.g., neovascularization of one or more tissues of the eye including, e.g., the cornea, retina, or choroid. Central to the present disclosure is the discovery that activation of a neuropeptide receptor such as, e.g., a melanocortin receptor (MC; such as, e.g., MC4), somatostatin (SST, such as, e.g., SST1-5) receptor, and/or an opioid receptor (such as, e.g., a delta, kappa, or mu opioid receptor) on pDCs of the eye can be used to reduce or limit neovascularization and/or inflammation in the eye. The methods and compositions of the invention are described further, as follows.

Plasmacytoid Dendritic Cells (pDCs)

Plasmacytoid dendritic cells (pDCs) are immune cells, which circulate in the blood and can also be found in peripheral lymphoid organs and some peripheral tissues. pDCs are bone marrow-derived innate immune cells that express Toll-like receptors (TLR) 7 and 9, PDCA-1, Siglec-H, and CD45R/B220, and, in mice, low levels of CD11c, which differentiates them from conventional dendritic cells (cDCs). In humans, pDCs are positive for blood-derived dendritic cell antigen (BDCA)-2 (CD303), BDCA-4 (CD304), and CD123. Upon activation, they produce large amounts of type 1 interferons (see, e.g., Tversky et al., Clin. Exp. Allergy 38(5):781-788, 2008; Asselin-Paturel et al., Nat. Immunol. 2(12):1144-1150, 2001; Nakano et al., J. Exp. Med. 194(8):1171-1178, 2001; Bjorck, Blood 98(13):3520-3526, 2001). As is discussed above, the present disclosure is based, in part, on the discovery that pDCs express neuropeptide receptors that, when activated, can increase production of angiostatic proteins that can inhibit neovascularization of avascular tissues such as, e.g., the cornea. Therefore, the present methods and compositions allow for activation of neuropeptide receptors expressed on pDCs, such as, e.g., MC (MC1-5) receptors, SST (SST 1-5) receptors, and/or opioid receptors (delta, kappa, or mu opioid receptors) to reduce neovascularization and/or inflammation in tissues of the eye in which the pDCs reside.

### Neuropeptide Receptor Agonists

The present disclosure provides neuropeptide receptor agonists that can be used in conjunction with the methods disclosed herein, as is discussed in detail below. The agonists can be administered as sole therapeutic agents or in combination with each other or other treatments that are known for the conditions described herein.

### Melanocortin Receptor Agonists

MC receptors are members of the rhodopsin family of 7-transmembrane G protein-coupled receptors. Five MC receptor family members are known to exist, including MC1, MC2, MC3, MC4, and MC5 receptors. MC receptors are activated by endogenous agonist melanocyte-stimulating hormones (MSH, such as, e.g., α-MSH, β-MSH, and γ-MSH), but may also be activated by synthetic agonists (e.g., small molecule agonists). MC receptor agonists that can be used in conjunction with the present disclosure include, but are not limited to agonist of MC1, MC2, MC3, MC4, or MC5 receptors. In particular embodiments, the MC receptor agonist is a MC4 receptor agonist. MC receptor agonists that may be used in conjunction with the methods and compositions described herein are provided in Table 1 below.

TABLE 1
Melanocortin receptor agonists

| | |
|---|---|
| Non-selective | α-MSH, β-MSH, γ-MSH, afamelanotide, |
| agonists | bremelanotide, melanotan II, modimelanotide, |
| | setmelanotide, agouti, agouti-related protein, |
| | ACTH, RY764 |
| MC1-selective | BMS-470,539 |
| MC4-selective | THIQ, PF-00446687, PL-6983 |
| Unknown | Alsactide, tetracosactide |
| selectivity | |

### Somatostatin Receptor Agonists

SST receptors are GPCRs that bind the ligand somatostatin, a small neuropeptide that functions in neural and immune signaling. Five SST receptor variants are known, including SST1, SST2, SST3, SST4, and SST5 receptors. SST receptor agonists that can be used in conjunction with the present disclosure include, but are not limited to agonist of SST1, SST2, SST3, SST4, or SST5 receptors. SST receptor agonists that may be used in conjunction with the methods and compositions described herein are provided in Table 2 below.

TABLE 2
Somatostatin receptor agonists

| | |
|---|---|
| Non- | Lanreotide, octreotide, octreotate, pasireotide, |
| selective | edotreotide, vapreotide, SST, cortistatin-14, CST17, |
| agonists | SRIF-14, SRIF-28 |
| SST4- | Cortistatin-14, CST-17, SRIF-14, SRIF-28, L-803,087, |
| selective | J-2156, NNC269100, H-c(DCys-Phe-LAgl(NβMe,benzoyl)- |
| | DTrp-Lys-Thr-Phe-Cys)-OH, veldoreotide |

### Opioid Receptor Agonists

Opioid receptors are a family of inhibitory GPCRs that naturally bind opioids as ligands and exhibit broad distribution in the brain, spinal cord, peripheral neurons, and digestive tract. Opioid receptors can be divided into four major subtypes, including delta (δ) opioid receptors (DORs; such as δ1 and δ2 receptors), kappa (κ) opioid receptors (KORs; such as κ1, κ2, and κ3 receptors), and mu GO opioid receptors (MORs; such as μ1, μ2, and μ3 receptors). Non-limiting examples of opioid receptor agonists that may be used in conjunction with the methods disclosed herein are provided in Table 3 below.

TABLE 3
Opioid receptor agonists

| | | |
|---|---|---|
| DOR | δ1 | Dynorphin A, dynorphin A (1-13), dynorphin A (1-8), dynorphin B, endomorphin-1, β- |
| agonists | | endomorphin, (Leu)-enkephalin, [Met]-enkephalin, α-neoendorphin, β-endorphin, |
| | | UFP512, AZD7268, BW373U89, DSLET, diprenorphine, DADLE, (−)-cyclazocine, |
| | | ADL5859, (−)-bremazocine, DPDPE, deltorphin II, etorphine, (D-Ala²)-deltorphin II, |
| | | BU08028, BW373U86, DSTBULET, ADL5747, ethylketocyclazocine, deltorphin II, |
| | | carfentanil, nalmefene, tramadol, cebranopadol, hydromorphone, nalorphine, |
| | | SNC80, normorphine, AR-M1000390, (−)-methadone, morphine, fentanyl, bilorphin, |
| | | dihydromorphine, etonitazene, nalbuphine, endomorphin-1, SCH221510, TAN-67, |
| | | ethylketazocine, pethidine |
| | δ2 | Dynorphin A, dynorphin A (1-13), dynorphin A (1-8), dynorphin B, endomorphin-1, β- |
| | | endomorphin, [Leu]-enkephalin, [Met]-enkephalin, α-neoendorphin, β-endorphin, |
| | | nalfurafine, ethyketazocine, enadoline, (−)-bremazocine, ethylketocyclazocine, |
| | | (−)-cyclazocine, butorphanol, etorphine, GR89696, enadoline, U69593, naloxone |
| | | benzoylhydrazone, MP1104, α-neoendorphin, HS665, β-neoendorphin, E2078, |
| | | spiradoline, asimadoline, ICI204448, tifluadom, cebranopadol, hydromorphone, |
| | | nalorphine, U69593, salvinorin A, BU08028, compound 3 [PMID: 23134120], |
| | | (−)-pentazocine, tramadol, normorphine, ADL5747, BW373U86, nalbuphine, ADL5859, |
| | | carfentanil, morphine, cebranopadol, dihydromoprihne, fentanyl, etonitazene, |
| | | SCH221510, UFP-512, hydrocodone, (−)-methadone, DAMGO, SR16835, bilorphin, |
| | | difelikefalin, HS665, [D-Ala²,F₅,Phe⁴]-dynorphin-(1-17)-NH₂, (−)-bremazocine, |
| | | spiradoline, U69593, nalbuphine, pethidine, AR-M1000390 |
| KOR | κ1, | Dynorphin A, big dynorphin, dynorphin A (1-13), dynorphin A (1-8), dynorphin B, |
| agonists | κ2, | endomorphin-1, β-endomorphin, (Leu)-enkephalin, (Met)-enkephalin, |
| | κ3 | α-neoendorphin, β-endorphin, nalfurafine, ethyketazocine, enadoline, (−)-bremazocine, |
| | | ethylketocyclazocine, (−)-cyclazocine, butorphanol, etorphine, GR89696, enadoline, |
| | | U69593, naloxone benzoylhydrazone, MP1104, E2078, spiradoline, asimadoline, |
| | | ICI204448, tifluadom, U50488, cebranopadol, hydromorphone, nalorphine, |
| | | salvinorin A, BU08028, (−)-pentazocine, tramadol, normorphine, ADL5747, |
| | | BW373U86, nalbuphine, ADL5859, carfentanil, morphine, dihydromorphine, |
| | | fentanyl, etonitazene, UFP-512, hydrocodone, (−)-methadone, DAMGO, SR16835, |
| | | bilorphin, difelikefalin, HS665, nalbuphine, pethidine, AR-M1000390 |
| MOR | μ1, | Dynorphin A, big dynorphin, dynorphin A (1-13), dynorphin A (1-8), dynorphin B, |
| agonists | μ2, | endomorphin-1, β-endomorphin, (Leu)-enkephalin, (Met)-enkephalin, |
| | μ2 | α-neoendorphin, β-endorphin, carfentanil, (−)-cyclazocine, butorphanol, sufentanil, |
| | | etonitazene, hydromorphone, ethylketocyclazocine, etorphine, fentanyl, DAMGO, |
| | | loperamide, (−)-methadone, cebranopadol, sufentanil, eluadoline, morphine, PZM21, |
| | | bilorphin, dihydromorphine, dynorphin-(1-1), normorphine, nalbuphine, |
| | | buprenorphine, eluxadoline, DADLE, hydrocodone, BU08028, cebranopadol, |
| | | DSLET, PL017, UFP-512, morphine, BW373U86, UFP-505, ADL5747, ADL5859, |
| | | SCH221510, SR16835, codeine, tepentadol, HS665, tramadol, PZM21, |
| | | levorphanol, BU72, methadone, pethidine, AR-M1000390, U-47700 |

The aforementioned agonists can be administered in amounts determined to be appropriate by those of skill in the art. Exemplary amounts of neuropeptide receptor agonists for administration are one or more drops (e.g., 1, 2, 3, 4, 5, or more drops) of a 0.05-10% w/v (e.g., 0.1-8%, 1-6%, 2-5%, or 3-4% w/v) solution or 0.5-1000 mg (e.g., 1-1000, 5-750, 10-500, 20-250, 30-100, 40-75, or 50-60 mg) per dose. Optionally, the neuropeptide receptor agonists are comprised within pharmaceutically acceptable compositions, such as ophthalmic compositions, as known in the art. Examples of such compositions are described below. The neuropeptide receptor agonists are included within these compositions in amounts sufficient to provide a desired dosage, using a desired volume (e.g., the volume of a drop from a standard eye dropper), as can be determined by those of skill in the art. The agonists can optionally be used in combinations (e.g., combinations of 1, 2, 3, 4, 5, 6, 7, 8, 9, 10, or more), with the combinations being from one, two, or all three types of general agonist types listed above.

### Identification of Subjects

Subjects that can be treated using the methods and compositions of the invention include those suffering from, or at risk for neovascularization and/or inflammation of the eye. The subjects include human patients (adults and children) who have or are at risk of developing a disease or condition of the eye, as is described herein.

Neovascularization is a common feature of many conditions, and may occur in tissues of the eye including, for example, the cornea, retina, or choroid. This process involves new blood vessel formation in abnormal locations, such as the cornea, a normally avascular tissue. Diseases that are characterized by corneal neovascularization include, for example, corneal infection, inflammation, autoimmune disease, limbal stem cell deficiency, neoplasia, dry eye disease, radiation, blepharitis, uveitis, keratitis, corneal ulcers, corneal graft rejection, glaucoma, rosacea, and lupus. Trauma, such as surgery, injury, burn (e.g., chemical burn), injury, and excessive or improper contact lens use, can also be characterized by neovascularization. Inflammation associated with ocular (e.g., corneal) neovascularization can result from bacterial and viral infection, Stevens-Johnson syndrome, graft rejection, ocular cicatricial pemphigoid, and degenerative disorders, such as pterygium and Terrien's marginal degeneration. Diseases or conditions that are characterized by retinal neovascularization include, for example, ischemic retinopathies, diabetic retinopathy, retinopathy of prematurity, retinal vein occlusions, ocular ischemic syndrome, sickle cell disease, radiation, and Eales' disease. Further, diseases or conditions that are characterized by choroidal neovascularization include, for example, inflammatory neovascularization with uveitis, macular degeneration, ocular trauma, trauma due to excessive or improper contact lens wear, sickle cell disease, pseudoxanthoma elasticum, angioid streaks, optic disc drusen, extreme myopia, malignant myopic degeneration, and histoplasmosis. Subjects having or at risk of developing any of the aforementioned disorders or conditions can be treated using the methods and compositions of the invention.

The cornea is the most densely innervated structure in the human body, and is therefore highly sensitive to touch, temperature, and chemical stimulation, all of which are sensed by corneal nerves. Corneal nerves are also involved in blinking, wound healing, and tear production and secretion. Damage to or loss of corneal nerves can lead to dry eyes, impairment of sensation, corneal edema, impairment of corneal epithelium healing, corneal ulcerations and erosions, and a cloudy corneal epithelium, among other conditions. Diseases or conditions characterized by corneal nerve degeneration or damage include, for example, dry eye disease, neurotrophic keratitis, corneal infections, excessive or improper contact lens wear, ocular herpes simplex (HSV), herpes zoster (shingles), chemical and physical burns, injury, trauma, surgery (including corneal transplantation, laser assisted in-situ keratomileusis (LASIK), penetrating keratoplasty (PK), automated lamellar keratoplasty (ALK), photorefractive keratectomy (PRK), radial keratotomy (RK), cataract surgery, and corneal incisions), abuse of topical anesthetics, topical drug toxicity, corneal dystrophies, vitamin A deficiency, diabetes, microbial keratitis, and herpetic keratitis (caused by, e.g., HSV-1). The methods and compositions of the invention can be used to prevent or treat any of the aforementioned diseases or conditions of the eye.

Patients having or at risk of developing diseases or conditions characterized by inflammation within the eye can also be treated using the methods and compositions of the invention. Thus, for example, patients having or at risk of the following diseases or conditions can be treated: episcleritis, scleritis, uveitis (e.g., anterior uveitis (including iritis and iridocyclitis), intermediate uveitis (including vitritis and pars planitis), posterior uveitis (including retinitis, choroiditis, chorioretinitis, and neuroretinitis), panuveitis (infectious) (including endophthalmitis), and panuveitis (non-infectious)), and retinal vasculitis.

### Compositions

Compositions of the invention include the agents (e.g., one or more, such as 1, 2, 3, 4, 5, 6, 7, 8, 9, 10, or more; see above) described herein (e.g., neuropeptide receptor agonists disclosed herein) in an ophthalmic administrable form. The compositions can thus include the agent in the form of, e.g., an aqueous solution, a gel, or a cream, which may include, e.g., one or more of the following excipients: glycerin, hydroxyethylcellulose (HEC), hydroxypropyl methylcellulose (HPMC), polyvinyl alcohol (PVA), carboxy methylcellulose (CMC), sodium chloride, polyvidone, polyethylene glycol, propylene glycol, hypromelloses, boric acid, sodium borate, sodium hyaluronate, and Hamamelis virginiana, optionally in combination with one or more preservative (e.g., benzalkonium (BAK), poloxamer 407, potassium sorbate, polyquad, sodium perborate, purite, cetrimide, hydroxypropyl guar, or polyquaternium).

In various specific examples, the compositions may include glycerin (0.1-3% v/v, e.g., 0.1%, 0.2%, 0.25%, 0.3%, 0.4%, 0.5%, 0.75%, 1.0%, 1.5%, 2.0%, 2.5%, or 3.0% v/v, or a range between any of these values), optionally in combination with propylene glycol (0.1-3% v/v, e.g., 0.1%, 0.2%, 0.25%, 0.3%, 0.4%, 0.5%, 0.75%, 1.0%, 1.5%, 2.0%, 2.5%, or 3% v/v, or a range between any of these values), polyethylene glycol (e.g., PEG400; 0.1-3% v/v, e.g., 0.1%, 0.2%, 0.25%, 0.3%, 0.4%, 0.5%, 0.75%, 1.0%, 1.5%, 2.0%, 2.5%, or 3.0% v/v, or a range between any of these values), and/or hypromelloses (0.1-3% w/v, e.g., 0.1%, 0.2%, 0.25%, 0.3%, 0.4%, 0.5%, 0.75%, 1.0%, 1.5%, 2.0%, 2.5%, or 3.0% w/v, or a range between any of these values). These compositions can optionally also include a preservative, e.g., BAK (0.001-0.05% w/v, e.g., 0.001%, 0.0025%, 0.005%, 0.01%, 0.025%, or 0.05% w/v, or a range between any of these values). In one specific example, the composition includes glycerin, polyethylene glycol (e.g., PEG400), and hypromelloses in, e.g., an amount as noted above (e.g., 0.2% v/v, 1% v/v, and 0.2% w/v, respectively).

In additional examples, the compositions include HEC (0.01-1% w/v, e.g., 0.01%, 0.025%, 0.05%, 0.07%, 0.1%, 0.5%, or 1% w/v, or a range between any of these values) and/or HPMC (0.1-1% w/v, e.g., 0.1%, 0.3%, 0.5%, 0.75%, or 1% w/v, or a range between any of these values, optionally in combination with dextran (e.g., dextran 70; 0.05%-1% w/v, e.g., 0.05%, 0.075%, 0.1%, 0.5%, or 1% w/v, ora range between any of these values). In particular examples, these compositions can optionally include one or more preservatives such, e.g., poloxamer 407 with potassium sorbate (0.05-0.5% w/v, e.g., 0.1% w/v), BAK (0.001-0.05% w/v, e.g., 0.001%, 0.0025%, 0.005%, 0.01%, 0.025%, or 0.05% w/v, or a range between any of these values), polyquad (0.0005-0.05% w/v, e.g., 0.0005%, 0.001%, 0.0025%, 0.005%, 0.01%, 0.025%, or 0.05% w/v, or a range between any of these values)), or sodium perborate (e.g., 0.001-5%, e.g., 0.01-1% or 0.05-0.35%). In various specific examples, the compositions can include 0.07% w/v HEC, poloxamer 407 (e.g., 0.001-5%, e.g., 0.01-1% or 0.05-0.35%), 0.01% w/v potassium sorbate; 0.3% w/v HPMC, 0.01% w/v BAK; 0.3% w/v HPMC, 0.0002 mL 50% w/v BAK; 0.3% w/v HPMC, 0.1% w/v dextran (e.g., dextran 70); 0.3% w/v HPMC, 0.1% w/v dextran 70, 0.001% w/v polyquad; 0.3% w/v HPMC, sodium perborate.

In other examples, the compositions include PVA (0.1-3% w/v, e.g., 0.1%, 0.25%, 0.5%, 0.75%, 1.0%, 1.25%, 1.4%, 1.5%, 1.75%, 2%, 2.5%, or 3% w/v, or a range between any of these values), optionally in combination with polyethylene glycol (0.1-3% w/v, e.g., 0.1%, 0.2%, 0.25%, 0.3%, 0.4%, 0.5%, 0.75%, 1.0%, 1.5%, 2.0%, 2.5%, or 3.0% w/v, or a range between any of these values) and/or povidone (0.1-3% w/v, e.g., 0.1%, 0.2%, 0.25%, 0.3%, 0.4%, 0.5%, 0.6% 0.75%, 1.0%, 1.5%, 2.0%, 2.5%, or 3.0% w/v, or a range between any of these values). These compositions can optionally also include a preservative, e.g., BAK (0.001-0.05% w/v, e.g., 0.001%, 0.0025%, 0.005%, 0.01%, 0.025%, or 0.05% w/v, or a range between any of these values). In various specific examples, the compositions can include 1.0% w/v PVA, 1.0% v/v polyethylene glycol, and 0.01% w/v BAK; 1.4% w/v PVA and 0.6% w/v povidone; 1.4% w/v PVA and 0.005% w/v BAK; or 0.5% w/v PVA and 0.6% w/v povidone.

In further examples, the compositions can include carboxymethylcellulose (CMC; 0.1-2% w/v, e.g., 0.1%, 0.25%, 0.5%, 0.75%, 1%, 1.25%, 1.5%, 1.75%, or 2% w/v, or a range between any of these values), optionally in combination with a preservative (e.g., purite, e.g., 0.001-5%, e.g., 0.01-1% or 0.05-0.35%).

In additional examples, the compositions can include sodium chloride (0.1-3% w/v, e.g., 0.1%, 0.25%, 0.5%, 0.64%, 0.75%, 0.9%, 1.0%, 1.25%, 1.4%, 1.5%, 1.75%, 2%, 2.5%, or 3% w/v, or a range between any of these values), optionally in combination with a preservative, e.g., BAK (0.001-0.05% w/v, e.g., 0.001%, 0.0025%, 0.005%, 0.01%, 0.025%, or 0.05% w/v, or a range between any of these values).

In further examples, the compositions can include polyvidone (1-10% w/v, e.g., 1%, 2.5%, 5%, 7.5%, or 10% w/v, or a range between any of these values) or povidone (1-10% w/v, e.g., 1%, 2.5%, 5%, 7.5%, or 10% w/v, or a range between any of these values), optionally in combination with a preservative, such as cetrimide (0.001-0.05% w/v, e.g., 0.001%, 0.0025%, 0.005%, 0.01%, 0.025%, or 0.05% w/v, or a range between any of these values).

Other exemplary compositions include polyethylene glycol (e.g., PEG400; 0.1-2% v/v, e.g., 0.1%, 0.25%, 0.4%, 0.5%, 0.75%, 1.0%, 1.5%, or 2.0% v/v, or a range between any of these values) and/or propylene glycol (0.1-2% v/v, e.g., 0.1%, 0.3%, 0.4%, 0.5%, 0.75%, 1.0%, 1.5%, or 2.0% v/v, or a range between any of these values), optionally in combination with a preservative such as, for example, hydroxypropyl guar (e.g., 0.001-5%, e.g., 0.01-1% or 0.05-0.35%) and/or polyquaternium-1 (e.g., 0.001-5%, e.g., 0.01-1% or 0.05-0.35%). In one specific example, such a composition may include 0.4% v/v polyethylene glycol 400, 0.3% propylene glycol v/v, hydroxypropyl guar (e.g., 0.001-5%, e.g., 0.01-1% or 0.05-0.35%), and polyquaternium-1 (e.g., 0.001-5%, e.g., 0.01-1% or 0.05-0.35%).

In further examples, the compositions may include boric acid (0.25-4% w/v, e.g., 0.25%, 0.5%, 0.75%, 1.0%, 1.3%, 2.0%, 2.5%, 3.0%, 3.5%, or 4% w/v, or a range between any of these values) and/or sodium borate (0.01-2% w/v, e.g., 0.01%, 0.05%, 0.1%, 0.32%, 0.5%, 1%, 1.5%, or 2% w/v, or a range between any of these values), optionally in combination with a preservative, e.g., BAK (0.001-0.05% w/v, e.g., 0.001%, 0.0025%, 0.005%, 0.01%, 0.025%, or 0.05% w/v, or a range between any of these values). A specific example of such a composition includes 1.3% w/v boric acid, 0.32% w/v sodium borate, and 0.01% w/v BAK.

In other examples, the compositions may include sodium hyaluronate (0.025-2.0% w/v, e.g., 0.025%, 0.05%, 0.1%, 0.25%, 0.5%, 0.75%, 1%, 1.25%, 1.5%, 1.75%, or 2% w/v, or a range between any of these values), optionally in combination with a preservative, e.g., BAK (0.001-0.05% w/v, e.g., 0.001%, 0.0025%, 0.005%, 0.01%, 0.025%, or 0.05% w/v, or a range between any of these values).

A further exemplary composition includes Hamamelis virginiana (e.g., 0.001-5%, e.g., 0.01-1% or 0.05-0.35%), optionally in combination a preservative, e.g., BAK (0.001-0.05% w/v, e.g., 0.001%, 0.0025%, 0.005%, 0.01%, 0.025%, or 0.05% w/v, or a range between any of these values).

The pH of the solutions described herein can be, e.g., 6.0-8.5, e.g., 6.5-8.0, 7.0-7.8, or 7.2-7.5, as determined to be appropriate by those of skill in the art.

In various examples, solutions at or close to the normal pH of the eye (pH 7.0-7.8) are used. Examples of such compositions include the following: 0.07% HEC, poloxamer 407 (e.g., 0.001-5%, e.g., 0.01-1% or 0.05-0.35%), 0.1% potassium sorbate; 0.3% HPMC, 0.01% BAK; 0.3% HPMC, 0.1% dextran; 0.3% HPMC, 0.1% dextran 70; 0.3% HPMC, 0.1% dextran 70, 0.001% polyquad; 0.5% CMC, purite (e.g., 0.001-5%, e.g., 0.01-1% or 0.05-0.35%); 0.9% sodium chloride, 0.0002 mL 50% BAK; 5.0% povidone, 0.005% centrimide; and Hamamelis virginiana (e.g., 0.001-5%, e.g., 0.01-1% or 0.05-0.35%), 0.005% BAK.

### Methods of Treatment

Neuropeptide receptor agonists may be administered to the eye of a subject to be treated according to the methods of the invention using methods that are known in the art for ophthalmic administration. Different routes of administration may be utilized, depending upon the part of the eye to be treated. For example, for treatment of a disease or condition of the cornea, direct topical application of a formulation (e.g., as described above) to the cornea can be used, optionally in combination with a treatment used to render the cornea permeable (e.g., by the application of topical anesthetic eye drops or by mechanical abrasion or removal of corneal epithelium). For treatment of a disease or condition of another part of the eye, e.g., the retina or the choroid, a different approach to administration may be selected. For example, intravitreal, sub-retinal, sub-conjunctival, or intracorneal injection may be utilized as determined to be appropriate by those of skill in the art.

Treatment according to the methods of the invention can be carried out using regimens that are determined to be appropriate by those of skill in the art based on factors including, for example, the type of disease, the severity of disease, the results to be achieved, and the age and general health of the patient. Treatment according to the methods of the invention thus can take place just once, or can be repeated (e.g., 2, 3, 4, 5, 6, 7, 8, 9, 10, or more times). In the case of multiple treatments, appropriate intervals between treatments can be selected by those of skill in the art. The invention thus includes, e.g., hourly, daily, weekly, monthly, bi-monthly, semi-annual, or annual treatments.

The methods of the invention can be used to treat a disease or condition of the eye by preventing or reducing corneal, retinal, or choroidal neovascularization in a subject by, for example, 10% or more (e.g., at least 10%, 20%, 30%, 40%, 50%, 60%, 70%, 80%, 90%, or 100%) as compared to the amount of neovascularization observed before treatment. For example, neovascularization and/or neovascularization can be reduced by 25%, 50%, 2-fold, 5-fold, 10-fold or more, or be eliminated. Improvements in neovascularization may be assessed clinically by fundus examination or Optical Coherence Tomography (OCT), as is understood in the art.

The methods of the invention can also be used to treat a disease or condition of the eye by preventing or reducing inflammation in the eye (e.g., cornea, retina, or choroid). For example, one way to modulate inflammation is to modulate an immune cell activity. This modulation can occur in vivo (e.g., in a human subject or animal model) or in vitro (e.g., in acutely isolated or cultured cells, such as human cells from a patient, repository, or cell line, or rodent cells). The types of cells that can be modulated include dendritic cells (e.g., pDCs, myeloid DCs/conventional DCs, or follicular DCs), T cells (e.g., peripheral T cells, cytotoxic T cells/CD8+ T cells, T helper cells/CD4+ T cells, memory T cells, regulatory T cells/Tregs, natural killer T cells/NKTs, mucosal associated invariant T cells, and gamma delta T cells), B cells (e.g., memory B cells, plasmablasts, plasma cells, follicular B cells/B-2 cells, marginal zone B cells, B-1 cells, regulatory B cells/Bregs), granulocytes (e.g., eosinophils, mast cells, neutrophils, and basophils), monocytes, macrophages (e.g., peripheral macrophages or tissue resident macrophages), myeloid-derived suppressor cells, natural killer (NK) cells, innate lymphoid cells (e.g., ILC1s, ILC2s, and ILC3s), thymocytes, and megakaryocytes. Inflammation can be modulated using the methods and compositions described herein by modulating immune cell activation (e.g., dendritic cell (e.g., pDC), macrophage, T cell, NK cell, ILC, B cell, neutrophil, eosinophil, or basophil activation). In certain embodiments, the inflammation is decreased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, or a range between any of these values, compared to before the administration of the neuropeptide receptor agonist. In certain embodiments, the inflammation is increased in the subject or cell between 5-20%, between 5-50%, between 10-50%, between 20-80%, between 20-70%, between 50-200%, between 100%-500%.

The effect of a neuropeptide receptor agonist on inflammation can also be assessed through measurement of secreted cytokines and chemokines in the eye. An activated immune cell (e.g., dendritic cell (e.g., pDC), T cell, B cell, macrophage, monocyte, eosinophil, basophil, mast cell, NK cell, ILC, or neutrophil) can produce pro-inflammatory cytokines and chemokines (e.g., IL-1β, IL-5, IL-6, IL-8, IL-10, IL-12, IL-13, IL-18, TNFα, and IFN-γ). Activation can be assessed by measuring cytokine levels in a blood sample, sample of a fluid obtained from the eye, lymph node biopsy, or tissue sample from a human subject, with lower levels of proinflammatory cytokines following treatment indicating decreased activation. Activation can also be assessed in vitro by measuring cytokines secreted into the media by cultured cells. Cytokines can be measured using ELISA, Western blot analysis, and other approaches for quantifying secreted proteins. Comparing results from before and after administration of a neuropeptide receptor agonist can be used to determine its effect.

In the case of prophylactic treatment, subjects at risk of developing a disease or condition of the eye, as described herein (e.g., subjects at risk for corneal, retinal, or choroidal neovascularization and/or inflammation due to a disease or condition of the eye), may be treated prior to symptom onset or when symptoms first appear, to prevent development or worsening of neovascularization, inflammation, degeneration, or damage. For example, in subjects already presenting with neovascularization and/or inflammation of the eye, further growth of vessels into presently avascular tissue can be prevented by the methods of the present invention. Similarly, in subjects already presenting with nerve damage or degeneration, further damage or degeneration can be prevented by use of the methods and compositions of the invention.

### Kits

The invention also provides kits that include a neuropeptide receptor agonist (e.g., a neuropeptide receptor agonist present in a pharmaceutically acceptable carrier or diluent; in e.g., a composition and/or amount as described herein) for use in preventing or treating diseases or conditions of the eye, e.g., as described herein. The kits can optionally include an agent or device for delivering the neuropeptide receptor agonist to the eye. For example, the kits may optionally include agents or devices for permeabilizing the cornea (e.g., topical anesthetic eye drops, tools for mechanically disrupting the corneal epithelium, and/or agents that enhance the uptake of the neuropeptide receptor agonist by cells). In other examples, the kits may include one or more sterile applicators, such as syringes or needles. Further, the kits may optionally include other agents, for example, anesthetics or antibiotics. The kit can also include a package insert that instructs a user of the kit, such as a physician, to perform the methods disclosed herein.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Expression of Angiostatic Factors by Murine and Human Plasmacytoid Dendritic Cells

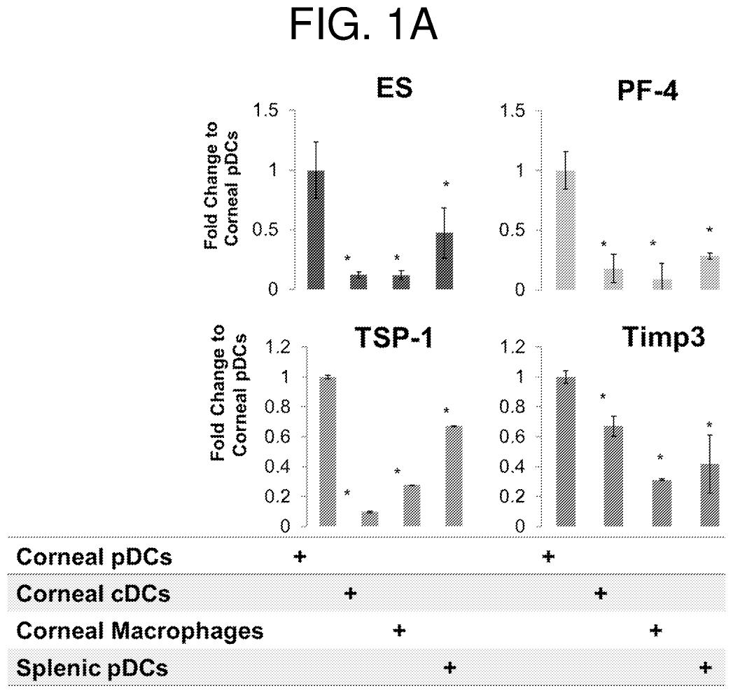

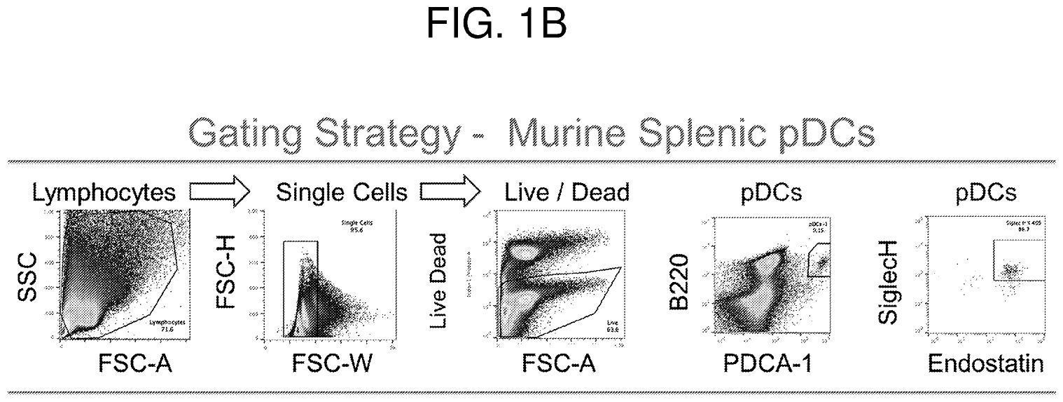

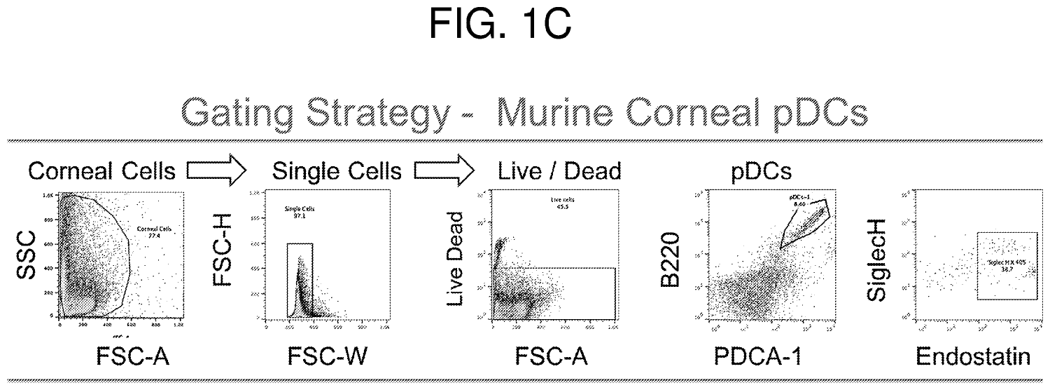

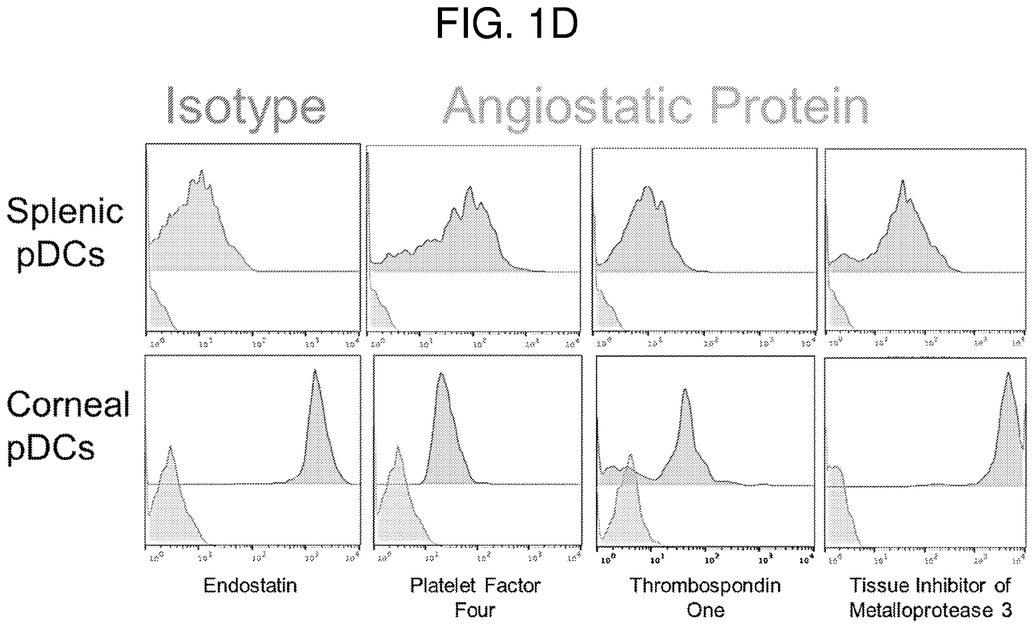

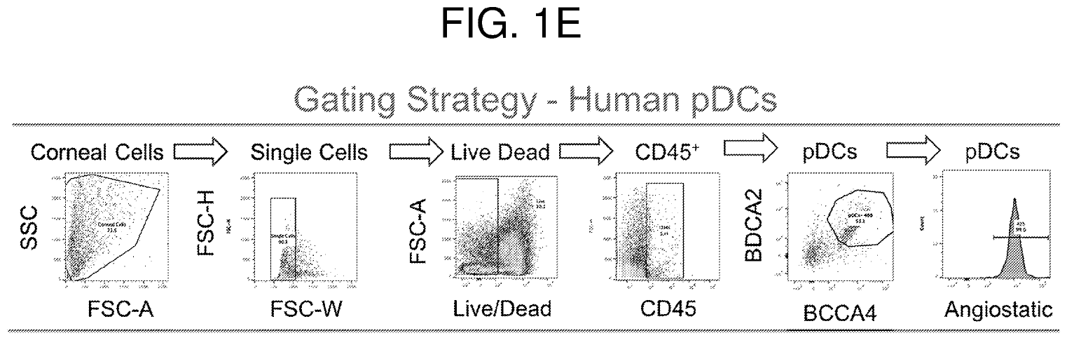

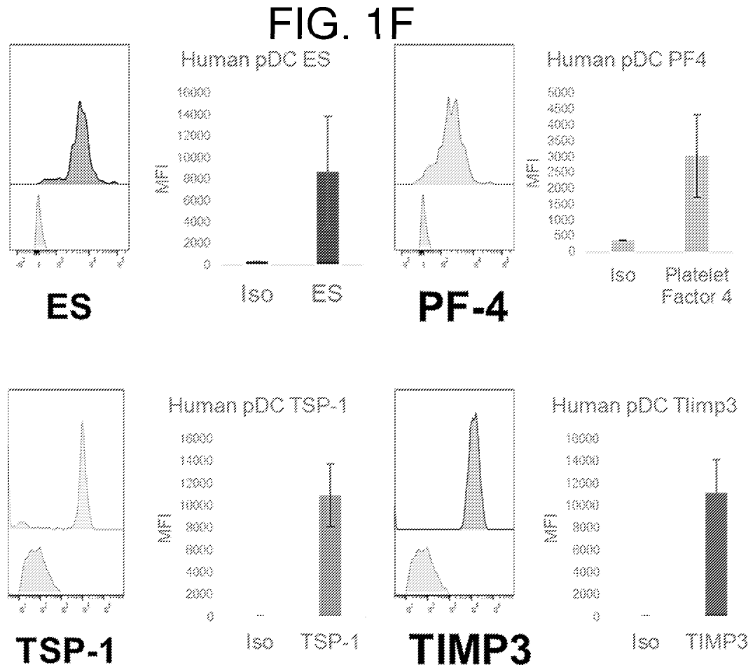

The cornea is the most densely innervated tissue in the body (Millodot, Ophthalmic Physiol. Opt. 4:305-318, 1984) and is endowed with resident Langerhans cells (LCs), conventional dendritic cells (cDCs), macrophages (Hamrah et al., Arch. Ophthalmol. 121:1132-1140, 2003; Hamrah et al., J. Leukoc. Biol. 74:172-178, 2003; Hamrah et al., Invest. Ophthalmol. Vis. Sci. 44:581-589, 2003; Hamrah et al., Invest. Ophthalmol. Vis. Sci. 43:639-646, 2002), and plasmacytoid dendritic cells (pDCs)(Sosnova et al., Stem Cells 23:507-515, 2005). Thus, the cornea is an ideal model to study neuro-immune interaction during homeostasis and the pathological induction of angiogenesis. To examine neuro-immune regulation of angiogenesis, the relative expression levels of the angiostatic molecules endostatin (ES)(Folkman, Nat. Med. 1:27-31, 1995; O'Reilly et al., Cell 88:277-285, 1997; Folkman, N. Eng. J. Med. 285:1182-1186, 1971; Lai et al., J. Biomed. Sci. 14:313-322, 2007; Ellenberg et al., Prog. Retin. Eye Res. 29:208-248, 2010), platelet factor 4 (PF4)(Sharpe et al., J. Natl. Cancer Inst. 82:848-853, 1990; Maione et al., Science 247:77-79, 1990; Kolber et al., J. Natl. Cancer Inst. 87:304-309, 1995), thrombospondin 1 (TSP-1)(Lawler, Curr. Opin. Cell Biol. 12:634-640, 2000; Lawler et al., Cold Spring Harb. Perspect. Med. 2: a006627, 2012; Lawler, J. Cell Mol. Med. 6:1-12, 2002; Cursiefen et al., Invest. Ophthalmol. Vis. Sci. 45:1117-1124, 2004), and tissue inhibitor of matrix metalloprotease three (TIMP3)(Qi et al., Nat. Med. 9:407-415, 2003; Lee et al., Mol. Vis. 15:2480-2487, 2009) were characterized in murine corneal resident leukocytes. All animal studies were conducted at Tufts Medical Center in agreement with the institutional animal care and use committee approved protocols. 6-8-week-old wild-type C57BL/6 animals were purchased from the Jackson Laboratory (Bar Harbor, Me., USA) or Charles River (Wilmington, Mass., USA) and housed in specific pathogen-free (SPF) facilities at Tufts Medical Center. Animals with corneal abnormalities were excluded from our studies. In all animal treatment groups, only the left eye was used unless otherwise noted. All experiments were carried out in accordance with the ARVO Statement for the Use of Animals in Ophthalmic and Vision Research. Murine pDCs express the pan leukocyte marker CD45, plasmacytoid dendritic cell antigen 1 (PDCA-1), sialic acid binding Ig-like lectin H (Siglec-H), and the B220 isoform of CD45R. Conventional dendritic cells, (cDCs) express the surface markers (CD45+, CD11c+), and macrophages express (IBA-1+, F4/80+). Corneal leukocytes were FACS sorted and levels of ES, PF4, TSP-1 and TIMP3 mRNA were quantified by qRT-PCR normalized to GAPDH and to corneal pDCs. Due to the low abundance of corneal pDCs (2% of corneal cells are CD45+ with 15-30% of CD45+ pDCs), data was pooled across corneas and single cell qRT-PCR. Corneal pDCs expressed significantly greater mRNA levels of ES, PF4, TSP-1, and TIMP3 when compared to corneal cDCs, corneal macrophages, and splenic pDCs (FIG. 1A). Utilizing flow cytometry, corneal and splenic pDC angiostatic protein expression was confirmed. In particular, corneas from C57BL/6 animals were harvested, pooled, and digested with collagenase D (2 mg/mL) (Roche) and Dnase (2 mg/mL) (MilliporeSigma) for 30 minutes at 37° C. and quenched with 10% fetal bovine calf serum in Ham's F-12 media. Spleens were isolated, mechanically strained using a 70-mm nylon mesh to yield single-cell suspension with erythrocytes lysed with ACK buffer (MilliporeSigma), and cells were resuspended in staining buffer (BD). Corneal cells and splenocytes were incubated with Brefeldin A (BD) for 4 hours. pDCs were incubated and blocked with 1% anti-CD16/CD32 FcR mAb (Bio X Cell) FC and stained using Live/Dead (Thermo Fisher), CD45 (Biolegend), PDCA-1 (Biolegend), Siglec-H (Biolegend) and B220 (Biolegend). Cells were fixed and stained with primary antibody for ES (Abcam), TSP-1 (Abcam), PF4 (Abcam) or TIMP3 (Abcam) and secondary AF488 (Abcam), AF405 (Abcam), or APC (Abcam) antibodies or appropriate corresponding isotype controls for 60-minute rocking at room temperature. Cells were washed with BD staining buffer and quantified using a BD LSR II flow cytometer. pDCs in splenocytes or pooled corneal cell suspensions were stained for pDCs surface markers (FIGS. 1B and 1C, respectively). Splenic (FIG. 1D, upper panel) and corneal pDCs (FIG. 1D, lower panel) expressed ES, PF4, TSP-1, and TIMP3. Despite differences in the expression of angiostatic molecules in corneal and splenic pDCs, this data suggests splenic pDCs may serve as a potential surrogate for corneal pDCs.

Phenotypically murine and human pDCs express divergent sets of surface markers as reviewed by Rogers et al. (Rogers et al., Am. J. Transplant. 13:1125-1133, 2013). Human pDCs express the pan leukocyte marker CD45, BDCA2 (CD303), and BDCA4 (neuropilin-1). To examine the possibility that murine but not human pDCs express angiostatic molecules, human corneal eye bank research samples for ES, PF-4, TSP-1 and TIMP3 were examined. Human corneal tissues were obtained and processed by Eversight Eyebank (Ann Arbor, Mich., USA) according to a standardized eye banking protocols, and procedures. Tissues which were deemed to be unsuitable for surgical use that had a normal endothelium, were included in our study. The exclusion criterion was tissue from donors with corneal neovascularization, or a history of diabetes, cancer, or keratitis. Human corneal cell suspensions were stained for pDCs (CD45+, BDCA2+, and BDCA4+; FIG. 1E) from three non-pair individual corneas. Phenotypic flow cytometry analysis of human corneal pDCs for ES, TSP-1, PF4, or TIMP3 from three individual human corneas (FIG. 1F) confirmed pDC angiostatic molecule expression. Taken together, these data show human and murine pDCs express angiostatic molecules and suggest a role for pDCs in regulating angiogenesis.

### Example 2: Corneal Nerves Regulate the Expression of Plasmacytoid Dendritic Cell Angiostatic Factor Expression Through the Melanocortin 4 Receptor

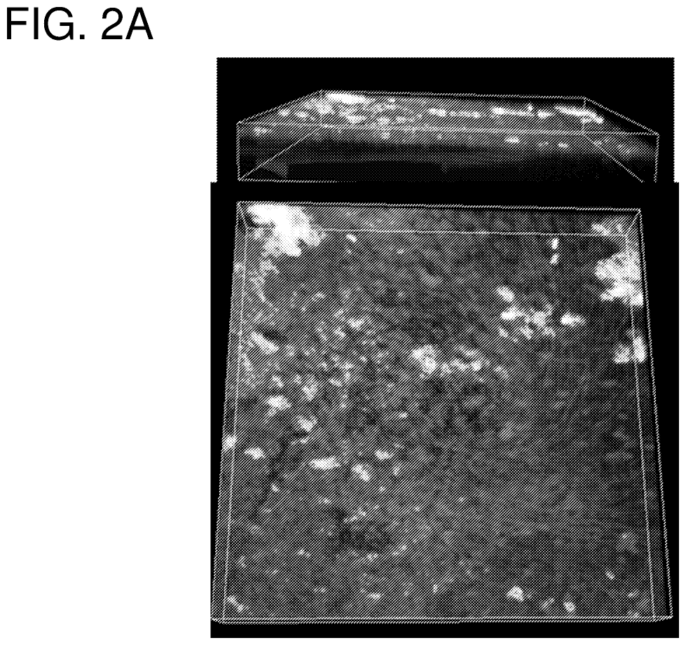

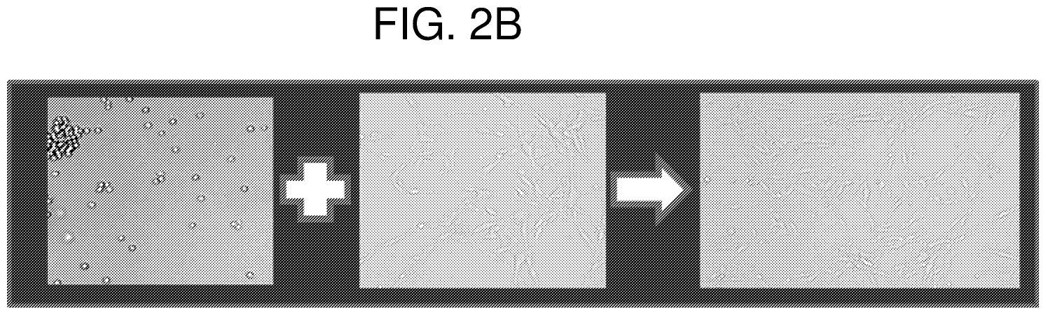

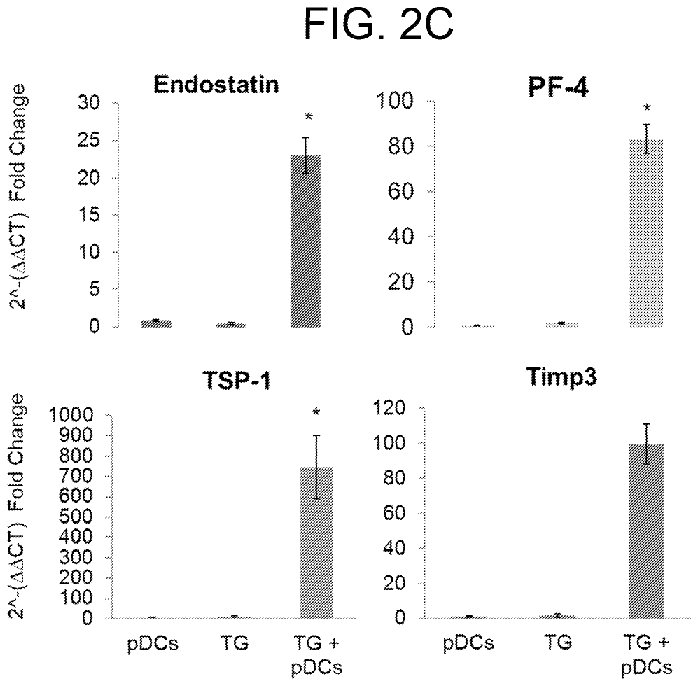

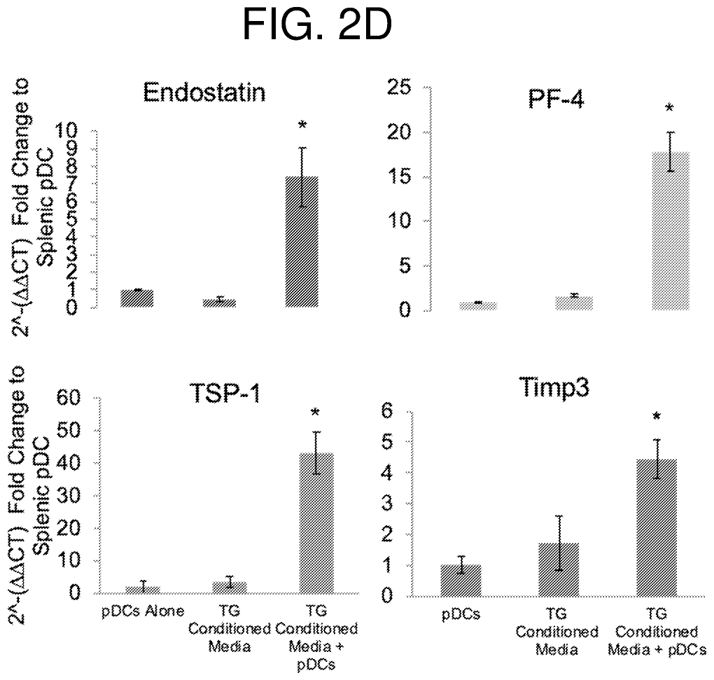

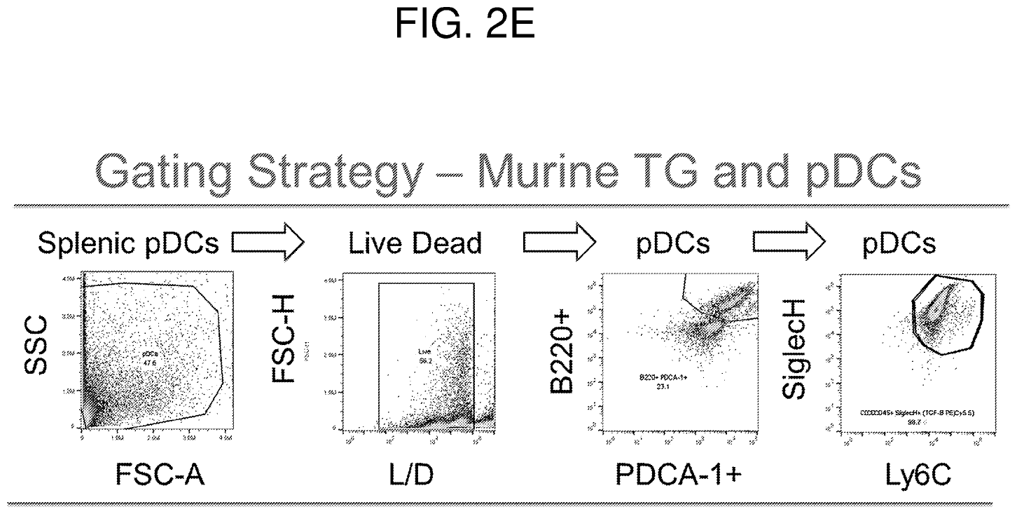

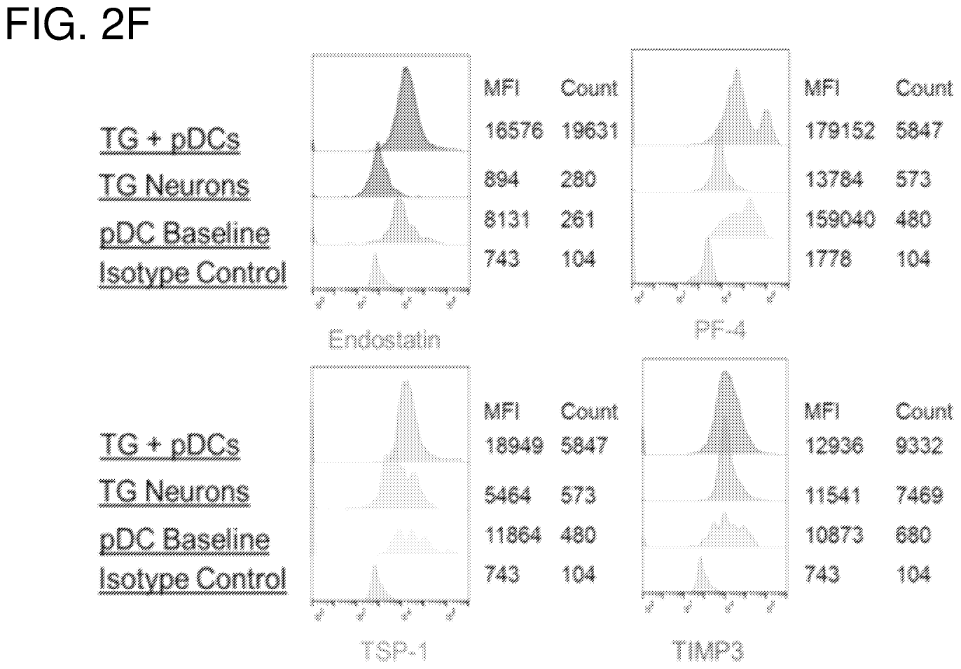

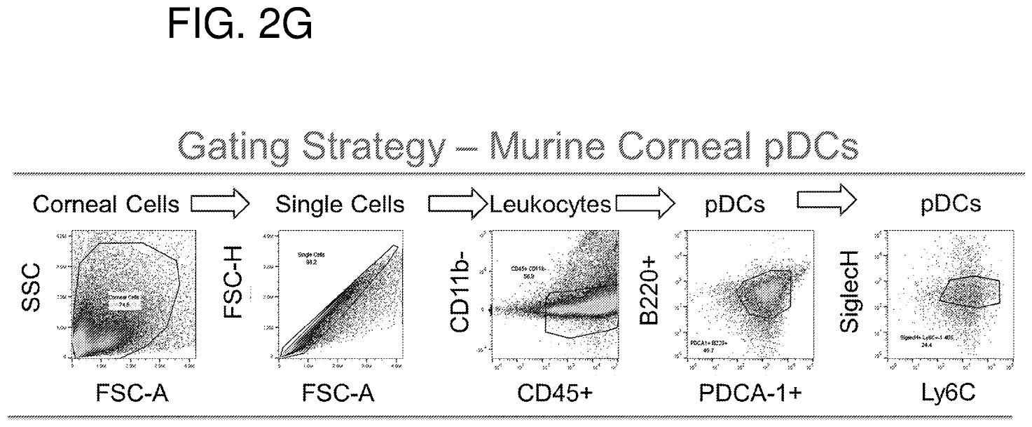

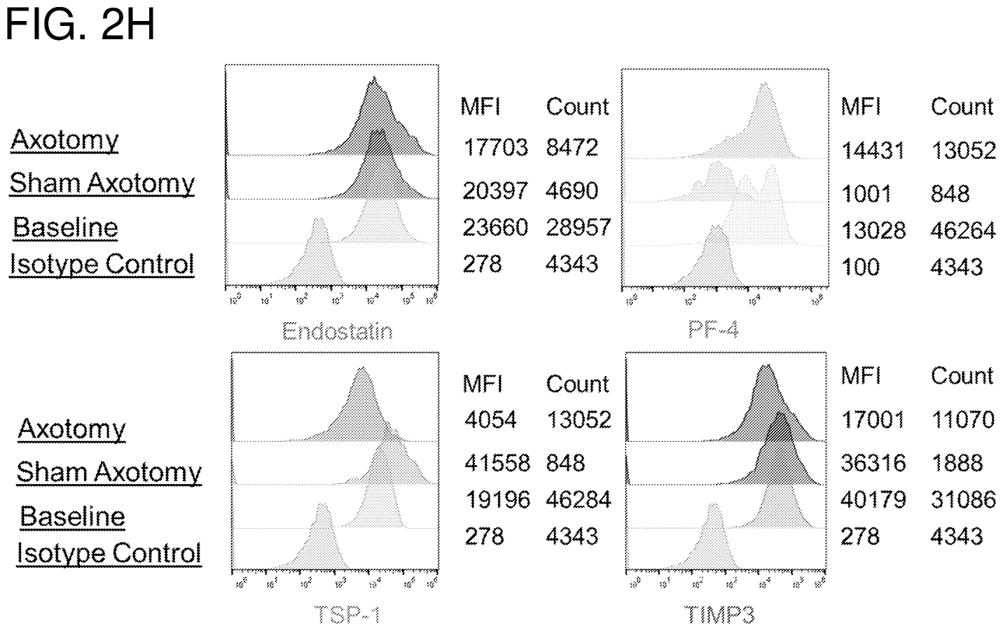

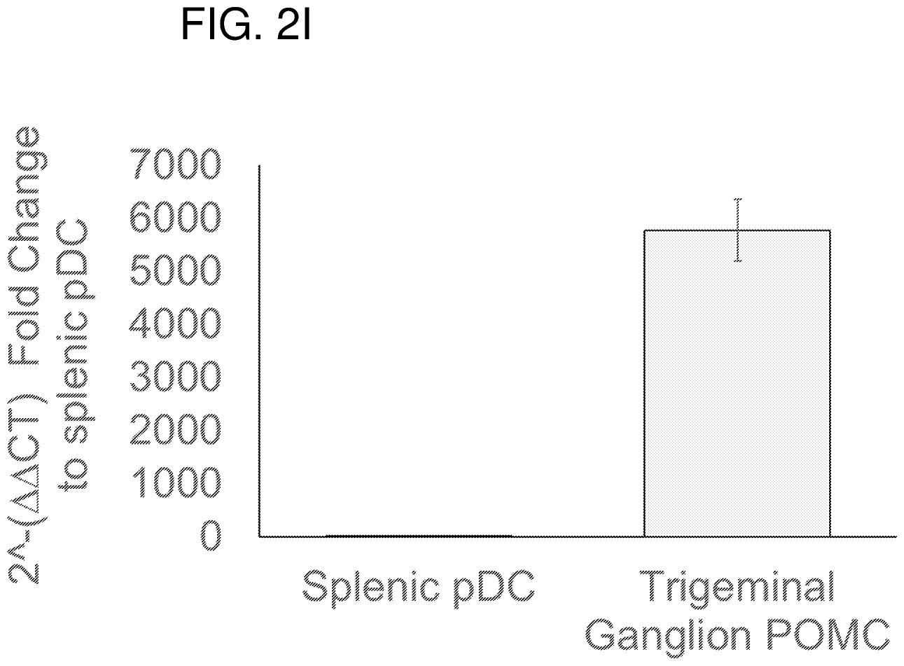

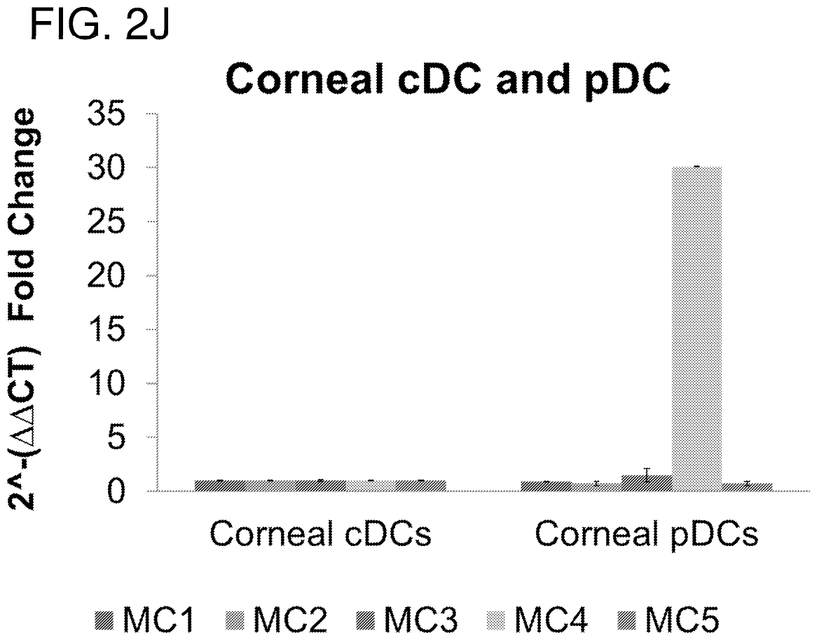

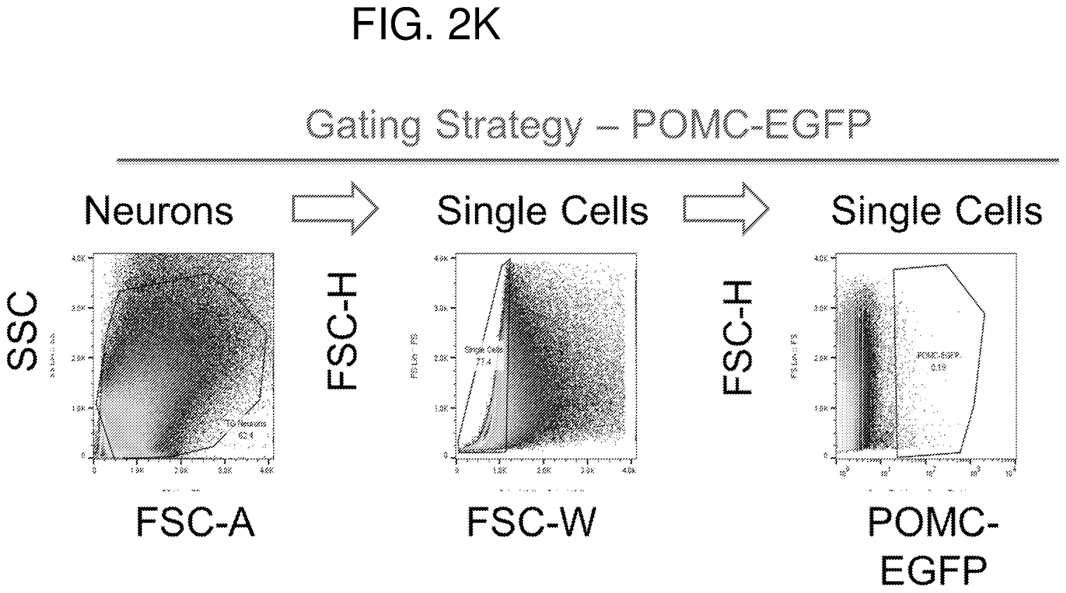

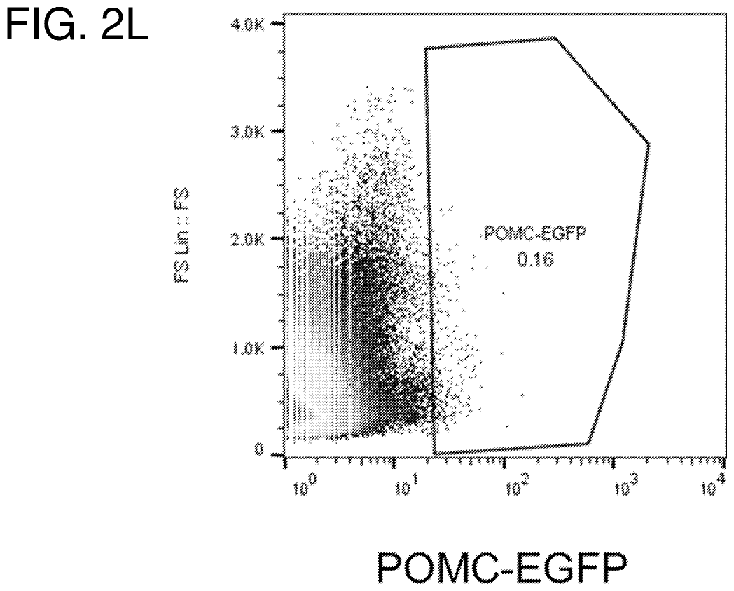

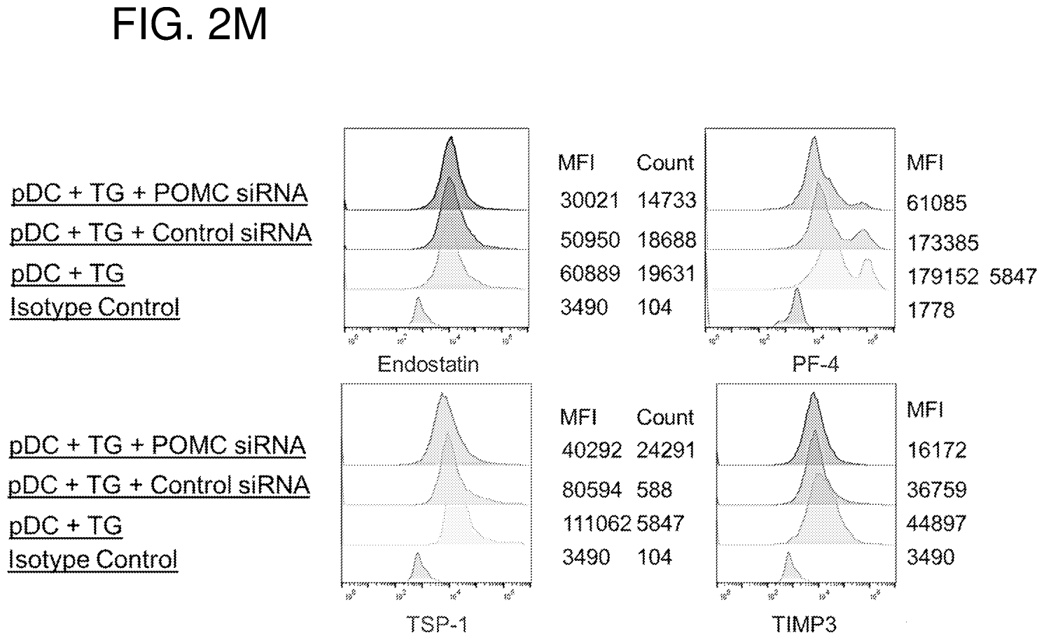

The potential regulation of the angiostatic activity of pDCs was examined. In vivo, corneal pDCs were intimately associated with the subbasal nerve plexus (FIG. 2A) and were not found in the stromal layer of the cornea. This association of pDCs with corneal nerves suggested corneal nerves may play a role in the regulation of pDCs. The sensory nerves that innervate the cornea are derived from the ciliary nerves of the ophthalmic branch of the trigeminal ganglion (TG) causing the cornea to be the most densely innervated tissue in the body (Rogers et al., Am. J. Transplant. 13:1125-1133, 2013). To test if corneal nerves impact pDC angiostatic activity, primary splenic pDCs were cultured with primary TG neuronal cell bodies as shown in bright field images (FIG. 2B). Specifically, TG neuronal cells were isolated as previously described (Sarkar et al., Invest. Ophthalmol. Vis. Sci. 54:5920-5936, 2013). Briefly, the TG was excised from postnatal day 10-14 C57BL/6 pups, pooled and digested with collagenase IV (2 mg/mL), Dispase (5 mg/mL) (Roche), and DNase (2 mg/mL) (Sigma). Neurons were separated from TG associated cells by Percoll (GE healthcare, Marlborough Mass.) gradient centrifugation. Neuronal enrichment was confirmed by qRT-PCR and flow cytometry for beta three tubulin. TG neuronal cells were isolated and cultured at a concentration of 1×10⁶ as outlined previously. Splenic pDCs were isolated and 1×10⁶ pDCs were incubated with 1×10⁶ TG nerve cells for 24 hours. Angiostatic mRNA was quantified as previously described. Conditioned Ham's F12 media with 10% FBS and 1% penicillin-streptomycin from trigeminal ganglion cells in cell culture for three weeks was incubated with 1×10⁶ splenic pDCs for 24 hours.

Co-Culture of FACS sorted pDCs with TG neurons significantly increased pDC ES, PF4, TSP-1, and TIMP3 gene expression by qRT-PCR when compared to pDCs or TG neurons alone (FIG. 2C). This induction of splenic pDCs suggested TG neurons may be regulating resident and infiltrating pDC ES, PF4, TSP-1 and TIMP3. To test if the induction was due to direct, or indirect contact of pDCs with TG neurons, pDCs were incubated with TG conditioned media. TG conditioned media increased pDC mRNA expression of ES, PF4, TSP-1, and TIMP3 by qRT-PCR when compared to pDCs or TG conditioned media alone (FIG. 2D). pDCs co-cultured with TG neurons expressed increased ES, TSP-1, PF4, or TIMP3 by flow cytometry (FIGS. 2E and 2F). Next, the contribution of the loss of corneal innervation to decreased pDC ES, PF4, TSP-1 and TIMP3 was examined in vivo. A well-established model of TG axotomy (Yamaguchi et al., PLoS One 8 e70908, 2013) and combined with flow cytometry were used to assess expression of angiostatic factors in pDCs. Specifically, animals are amnestied, and the surgical area was shaved. An incision was made with two weighed sutures placed to obtain a clear surgical field and allow the eye globe to be dislocated easily. The lateral fornix and soft tissue preparation were incised. After rotating the eye nasally gently by holding the limbal conjunctiva, the nasal fornix was pushed using blunt curved forceps. The eye globe was rotated to control the eye position to enable visualization of the optic nerve and the branches of the trigeminal nerve. The branches of the trigeminal nerve were removed, the weights were removed followed by tarsorrhaphy. Axotomy of TG inputs to the cornea produced a decrease in pDC ES, PF4, TSP-1, and TIMP3 expression 24 hours after corneal axotomy (FIGS. 2G and 2H). Taken together, these data show TG neurons can induce pDC angiostatic protein expression.

### Example 3: Plasmacytoid Dendritic Cells Express Neuropeptide Receptors

Neurons and some leukocytes are known to share expression of neuropeptide receptors and their corresponding ligands (Goetzl et al., FASEB J. 6:2646-2652, 1992; Ho et al., J. Immunol. 159:5654-5560, 1997). The impact of TG neurons on pDCs suggested pDCs may respond to TG derived neuropeptides. Previous studies have characterized the presence of the pro-opiomelanocortin (POMC) derivative a-MSH71 in the cornea. Neuropeptide ligand expression by isolated TG neurons and splenic pDCs was characterized. qRT-PCR revealed greater expression of POMC by TG neurons compared to splenic pDCs (FIG. 2I). This suggested a paracrine signaling between TG neurons and pDCs and not autocrine signaling between pDCs. Previous studies have shown an angiostatic activity of POMC derivatives on endothelial cells (Weng et al., Biochim Biophys Acta 1840:1850-1860, 2014) and for MC receptors in the retina (Rossi et al., Mediators Inflamm. 2016:7368389, 2016). These suggested a possible role for TG derived POMC and melanocortin receptors in pDC angiostatic molecule expression. Using qRT-PCR on FACS sorted pDCs, the relative mRNA expression of pDC MC receptor isoforms was quantified and compared to expression in corneal cDCs. pDCs expressed greater levels of the MC4 receptor compared to other MC receptor isoforms (FIG. 2J). TG expression of POMC was confirmed (FIG. 2K). Expression of the MC4 receptor was confirmed on human pDCs (FIG. 2L). To test the specificity of the MC4 receptor for TG mediated induction of pDC ES, PF4, TSP-1, and TIMP3, co-cultures of pDCs with TG neurons transfected with siRNA against POMC or a control siRNA were carried out. Flow cytometry revealed that POMC siRNA reduced pDC ES, PF4, TSP-1, and TIMP3 MFI (FIG. 2M). Together, these data show neuronal modulation of pDC angiostatic activity through the MC4 receptor in murine and human pDCs.

### Example 4: Melanocortin 4 Receptor Agonist Increases Plasmacytoid Dendritic Cell Expression of Angiostatic Factors and Reduces Corneal Neovascularization

### THIQ Induces pDC Angiostatic Molecule Expression

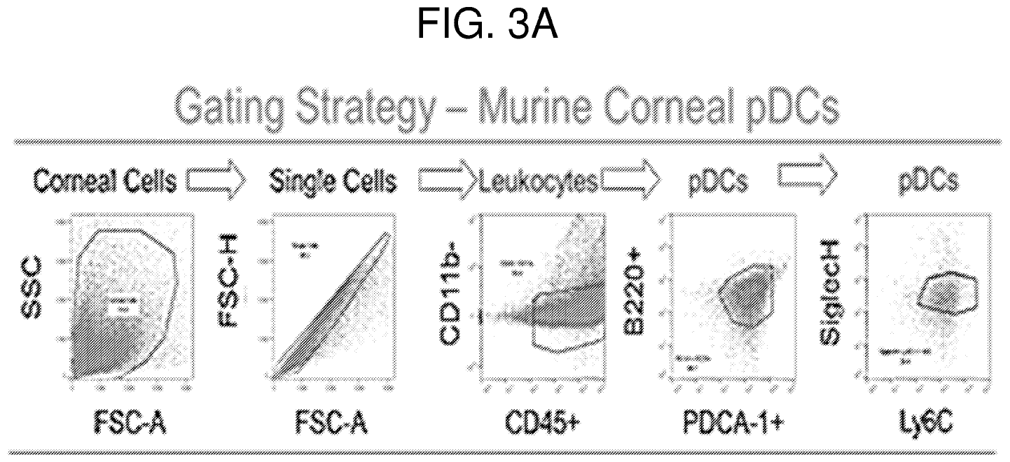

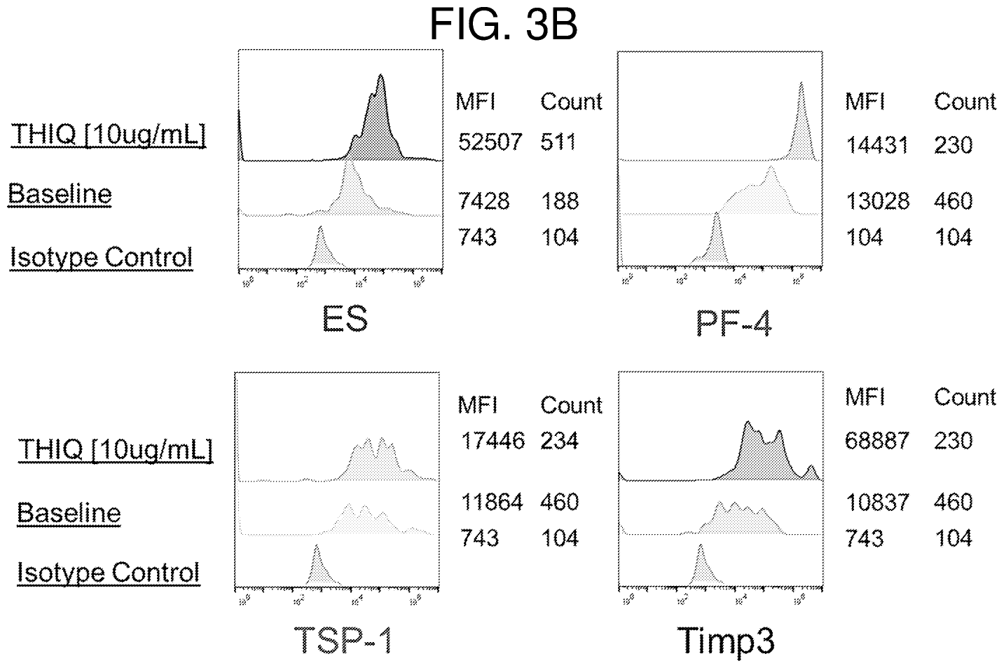

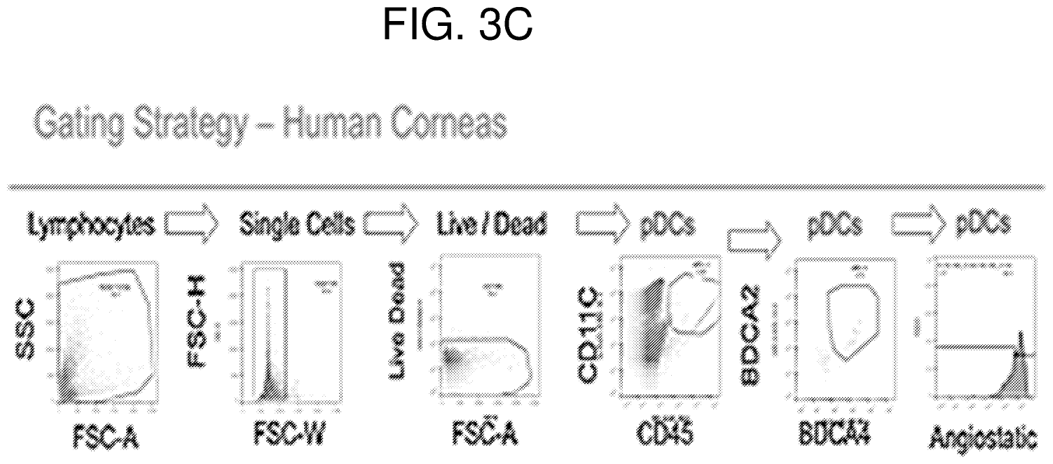

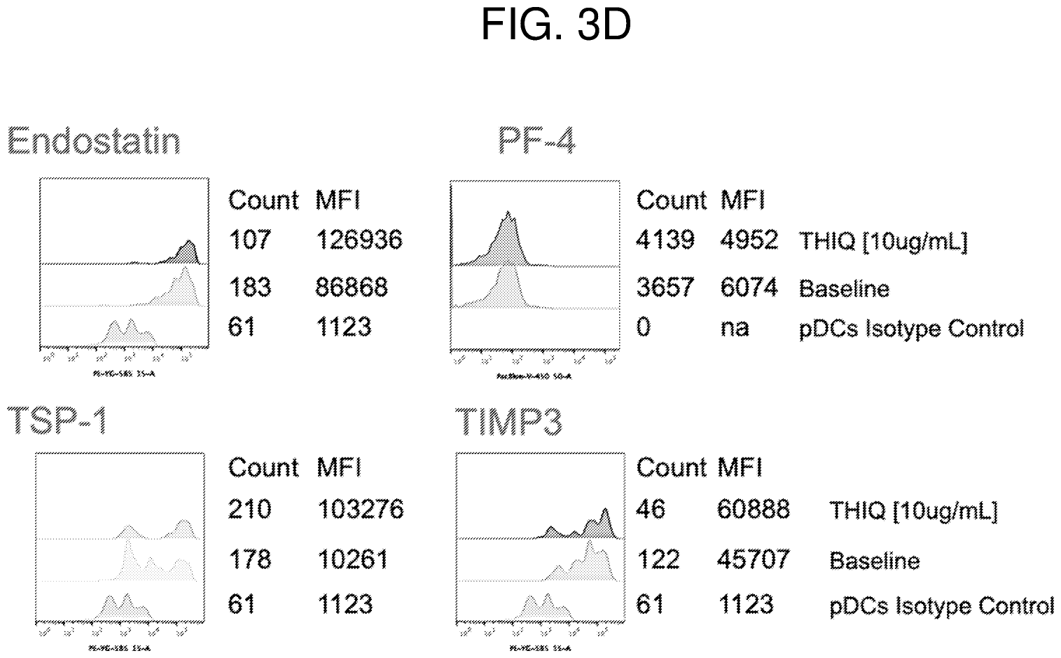

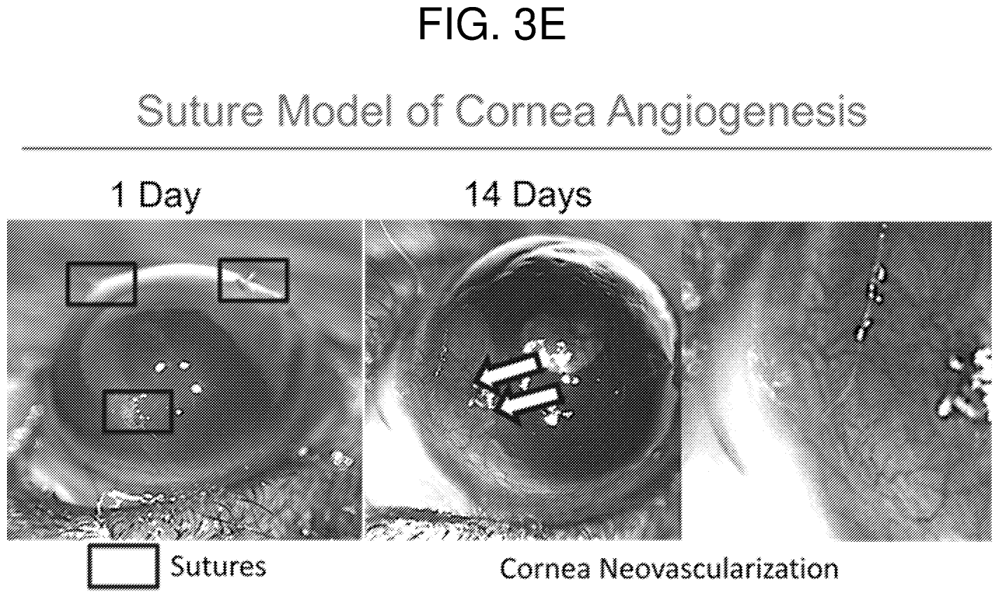

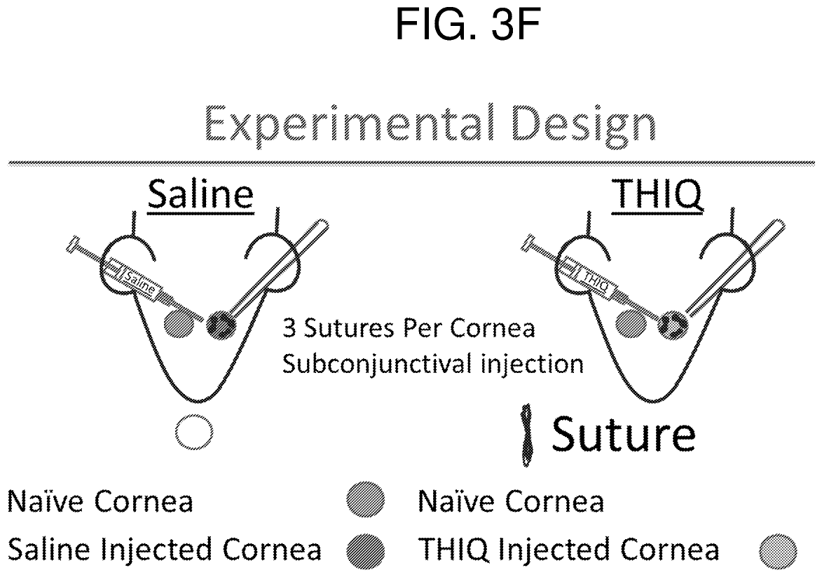

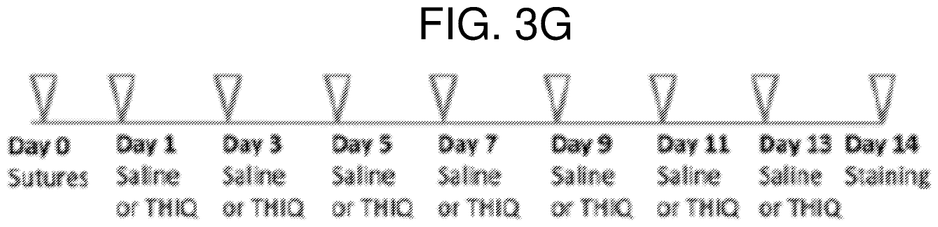

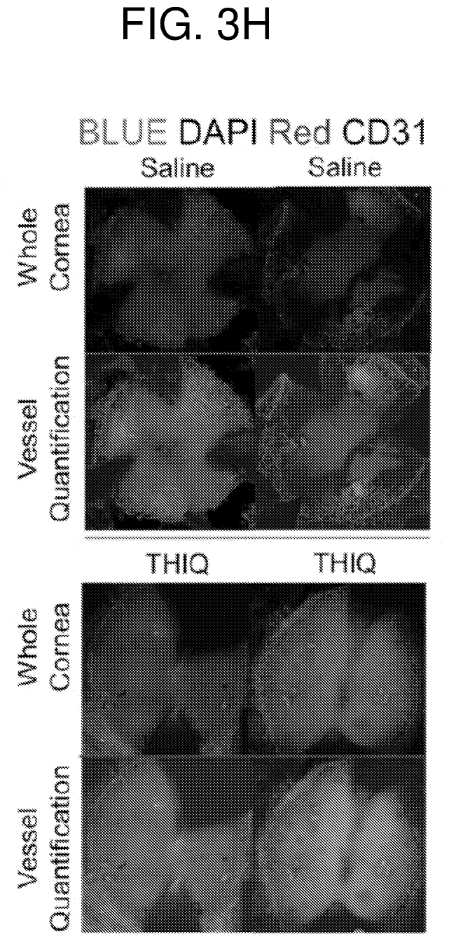

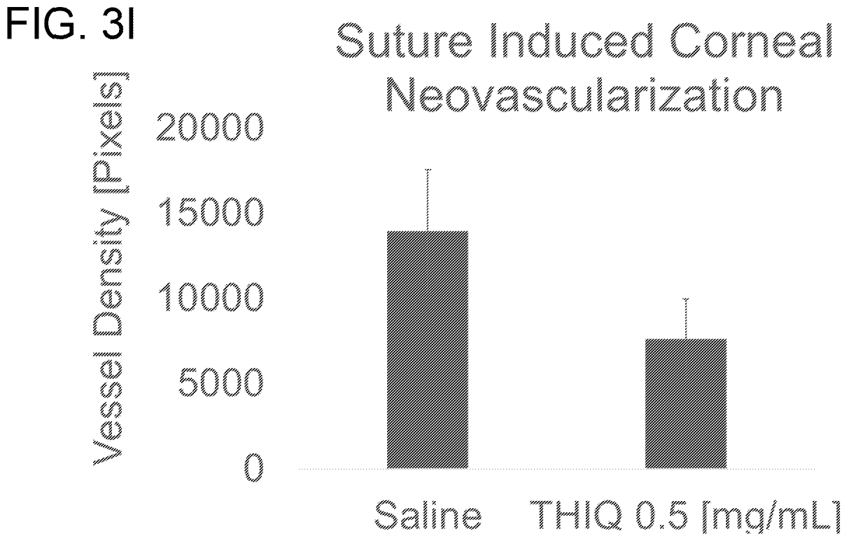

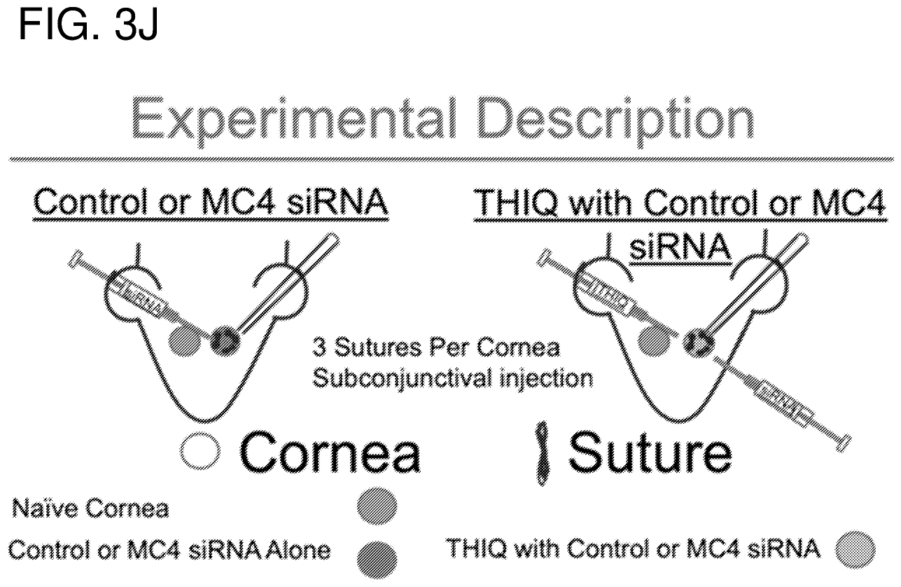

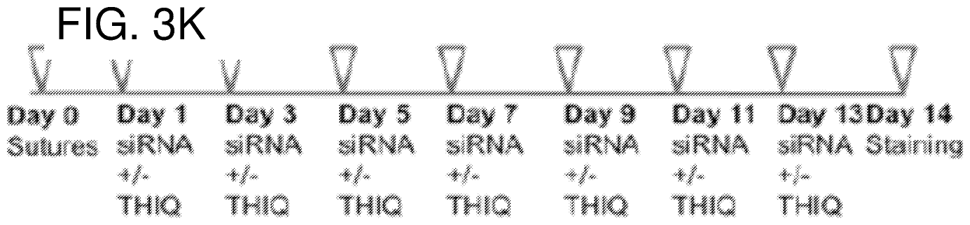

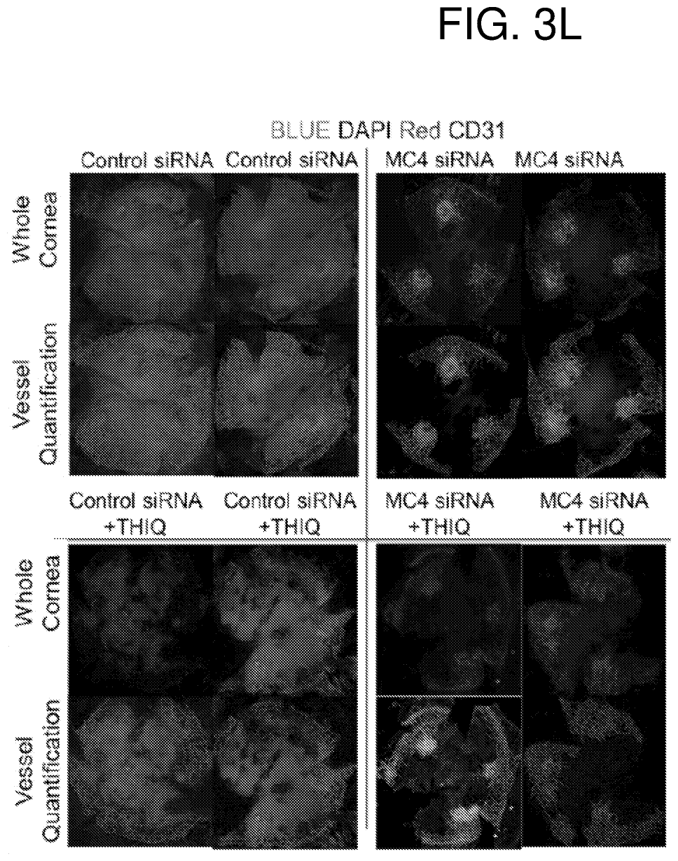

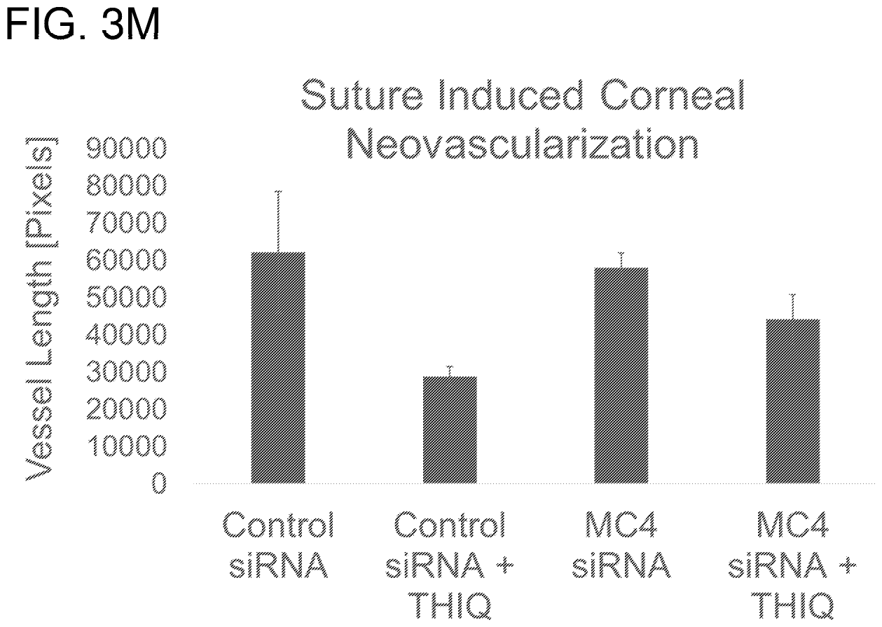

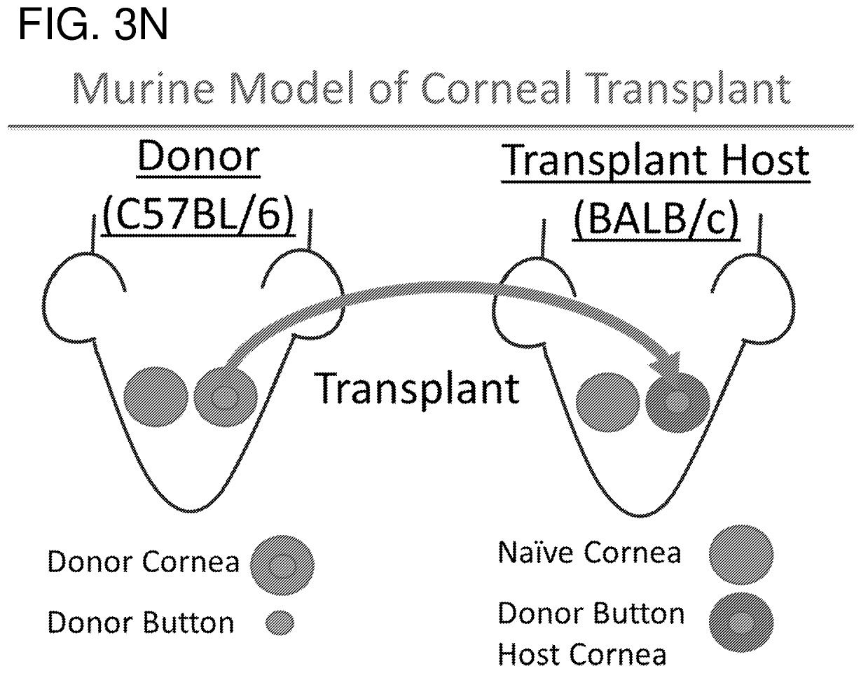

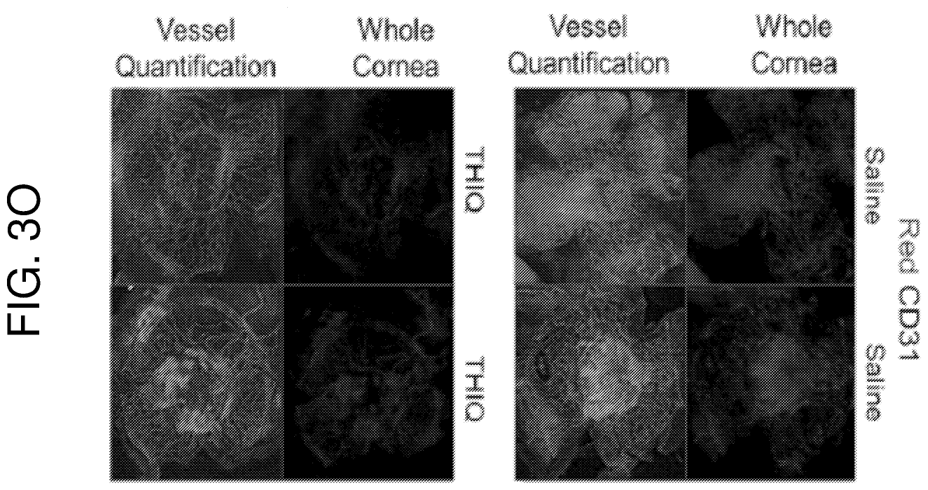

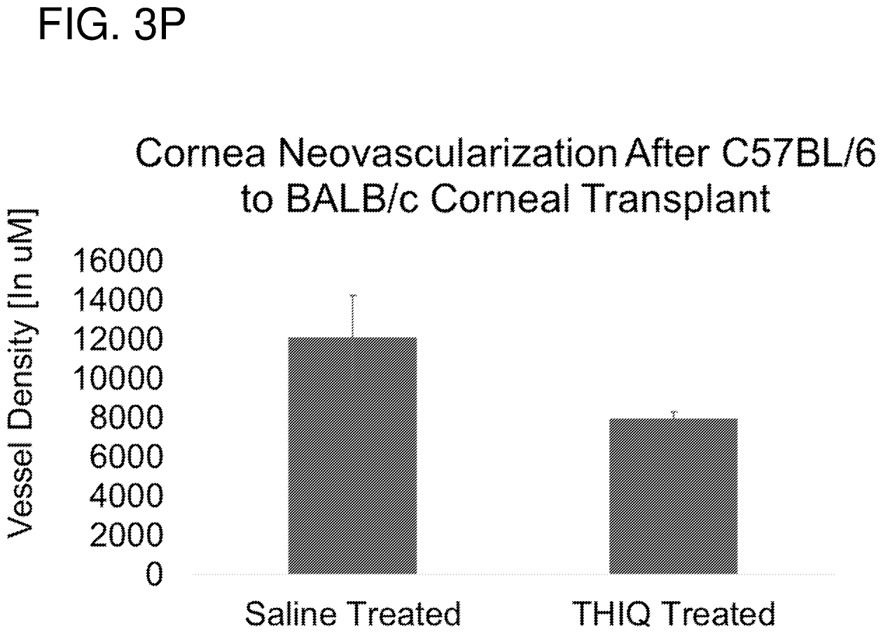

To examine the in vivo impact of pDC MC4 receptor activation, the selective agonist THIQ ((3R)—N-[(2R)-3-(4-chlorophenyl)-1-[4-cyclohexyl-4-(1,2,4-triazol-1-ylmethyl)piperidin-1-yl]-1-oxopropan-2-yl]-1,2,3,4-tetrahydroisoquinoline-3-carboxamide (Muceniece et al., Basic Clin. Pharmacol. Toxicol. 101:416-420, 2007; Sebhat et al., J. Med. Chem. 45:4589-4593, 2002) was utilized. The ability of THIQ (10 ug/mL) to impact murine and human pDCs was tested. Flow cytometry revealed that THIQ increased murine (FIGS. 3A and 3B) and human (FIGS. 3C and 3D) ES, PF4, TSP-1, and TIMP3 mean fluorescence intensity (MFI).

### Subconjunctival THIQ Injections Reduce CNV

A well-established tissue model to examine in vivo angiogenesis is a chemical (bFGF, VEGF) or physical (suture) stimulus in the cornea. Corneal Neovascularization (CNV), develops when a stimulus causes new blood vessels to extend into the cornea from the vascular limbus. To examine the impact of MC4 activation on CNV in vivo, a suture induced model of CNV19 was utilized (FIG. 3E). C57BL/6 animals received three sutures in one cornea while the contralateral cornea was left untouched (FIG. 3F). Specifically, three interrupted intrastromal sutures (Nylon 11-0 taper point; Surgical Specialties) were placed to induce corneal neovascularization as previously described (Streilein et al., Invest. Ophthalmol. Vis. Sci. 37:413-424, 1996). To minimize pain, Buprenorphine-SR Lab (0.1 mg/kg) was administered subcutaneously prior to surgery. Briefly, C57BL/6 animals were anesthetized with xylazine (20 mg/kg) and ketamine (100 mg/kg) and sutures were placed in the cornea in a triangle pattern. To prevent infection, erythromycin ophthalmic ointment (USP, 0.5%; Bausch & Lomb) was applied to the cornea. Animals were monitored three times daily for three days for signs of adverse effects. Through subconjunctival injection, animals received either sterile saline, or sterile THIQ every other day for 14 days (FIG. 3G). Specifically, adult C57BL/6 mice were anesthetized with isoflurane and injected subconjunctivally with 5 μL of saline (MilliporeSigma) or THIQ (Tocris) (1 mg/mL) twice every other day for two weeks. For MC4 siRNA (Santa Cruz) experiment, animals received 8 μM initially, then received 4 μM every other day per eye for two weeks (Berger et al., PLoS Pathog. 9: el 003457, 2013). For siRNA experiment, to limit volume injected into the cornea, THIQ was resuspended in siRNA solution. After two weeks, animals were sacrificed, and corneas were harvested for confocal whole mount staining. Specifically, whole corneas were excised from naïve, or corresponding treatment groups, from 6-8-week-old adult C57BL/6 mice. Corneas were fixed in chilled acetone for 15 minutes at −20° C., washed 3 times with staining buffer, and blocked with 1% anti-CD16/CD32 FcR mAb (Bio X Cell) FC in 3% bovine serum albumin (MilliporeSigma) to limit non-specific staining. Cells were stained overnight at 4° C. for CD31(Biolegend), CD45, PDCA-1, βIII-tubulin (Biolegend), and DAPI then imaged by confocal microscopy on a SP8 (Leica Microsystems). The entire corneal thickness was measured including both the stromal, and subbasal nerves (˜120-micron thickness). The entire corneal area was quantified by mosaic imaging of 25-40 fields of view. Blood vessels were quantified by CD31 (Abcam) staining and traced with ImageJ. Each image is approximately 25-40 fields of view with a 10× objective. CNV was induced in saline treated animals (FIG. 3H, upper panel). Suture induced CNV was reduced by 46% after subconjunctival injection of THIQ (10 ug/mL) (FIG. 3H (lower panel) and 3I).

To confirm the role of MC4 on inhibition of CNV after THIQ treatment, siRNA against POMC was injected prior to treatment with THIQ. C57BL/6 animals received three sutures and were injected every other day for two weeks with either, control siRNA, control siRNA with THIQ, siRNA against the MC4 receptor, or siRNA against MC4 with THIQ (FIGS. 3J and 3K). CNV was quantified as described previously. No statistical difference in CNV was found between control siRNA, MC4 siRNA, and MC4 siRNA with THIQ. (FIGS. 3L and 3M).

Having established a role for THIQ and MC4 in reducing CNV in vivo, a disease model of corneal transplant (She et al., Ophthalmic Surg. 21:781-785, 1990) was utilized. In particular, a standardized protocol for murine orthotopic corneal transplantation was utilized with modifications as previously described (Hamrah et al., Invest. Ophthalmol. Vis. Sci. 48:1228-1236, 2007). Briefly, the donor cornea button was prepared from a wild-type C57BL/6 mouse. The cornea was excised with Vannas scissors, (Fine Science Tools, CA) and placed into chilled phosphate-buffered saline (PBS). BALB/c animals were used as the corneal recipient. The recipient graft bed was prepared by excising a 1.5 mm site in the central cornea. The donor button was then placed onto the corneal bed of recipients and secured with eight interrupted 11-0 nylon sutures (Accutome, Pa.). Antibiotic ointment was applied, followed by a 24-hour tarsorrhaphy with 8-0 nylon sutures (Accutome). Graft sutures were removed on day 7, and animals were sacrificed on day 14. Corneas were excised and stained for CNV. In allogenic corneal transplant, immune infiltration and inflammation as well as corneal CNV leads to graft rejection. Donor C57BL/6 corneal buttons were implanted into host BALB/c corneal beds (FIG. 3N). This led to an HLA mismatch resulting in inflammation, CNV, and ultimately graft rejection. Through subconjunctival injection, animals received either sterile saline, or sterile THIQ (10 ug/mL) every other day for 14 days. Transplanted corneal whole mounts were collected and quantified for CD31 staining as previously described. THIQ reduced CNV in C57BL/6 to BALB/c corneal transplants (FIGS. 30 and 3P).

### Example 5: Activation of Melanocortin 4 Receptors on Plasmacytoid Dendritic Cells Increases Production of Angiostatic Factors

Previous studies have shown the MC4 receptor to couple to all three major classes of G proteins, Gs, Gi/o, and Gq depending on cell type (Tao, Endocr. Rev. 31:506-543, 2010). To test pDC MC4 signaling, FACS-sorted pDCs were incubated with THIQ (10 ug/mL) for 5, 15, or 30 minutes. Protein kinase C (PKC) isoform phosphorylation was examined by phospho-PKC specific antibodies and immunoblotting. Specifically, Splenic pDCs were FACs sorted as previously described. Five hundred thousand pDCs were used per condition and lysed directly with chilled 1× radioimmunoprecipitation assay (RIPA) buffer or 1× Laemmli Buffer on ice. Samples were diluted in NuPAGE sample loading buffer (ThermoFisher) and 1× NuPAGE Sample Reducing Agent (ThermoFisher) and heated to 70° C. for 10 minutes. Protein lysates were resolved on a 10% Bis-Tris SDS gel (ThermoFisher) and transferred to a nitrocellulose membrane (ThermoFisher). Membranes were blocked with odyssey blocking buffer (Licor) for one hour and incubated with protein specific primary antibodies (1:100 or 1:200) overnight at 4° C. Membranes were washed with blocking buffer and secondary antibodies were diluted (1:5000 or 1:15000) and incubated with membranes for 60 minutes at room temperature. Membranes were imaged using an Odyssey® CLx Imaging System (Licor). Image analysis was done using Image Studio software. Protein sampling kits for PKC, and NF-κB signaling antibodies were purchased from Cell signaling (Phospho-PKC Antibody Sampler Kit #9921 and NF-κB Pathway Sampler Kit #9936).

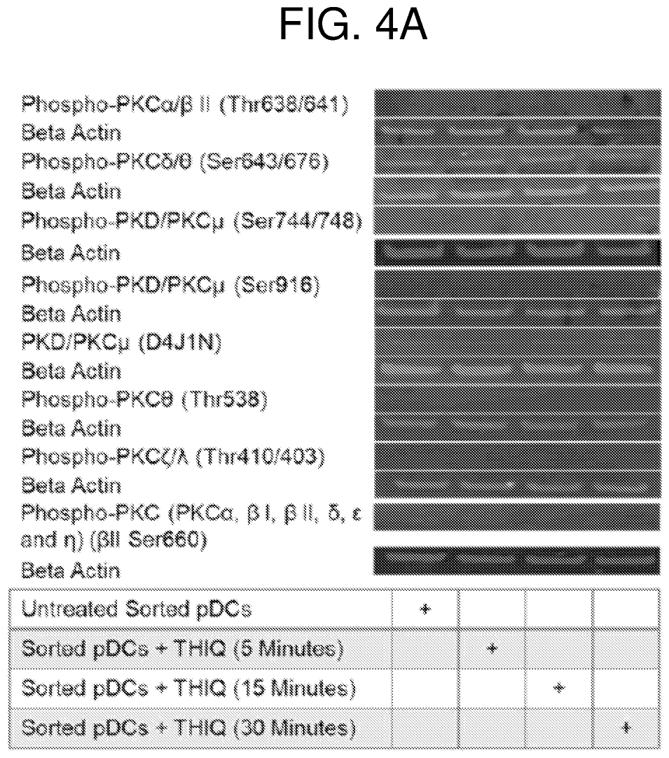

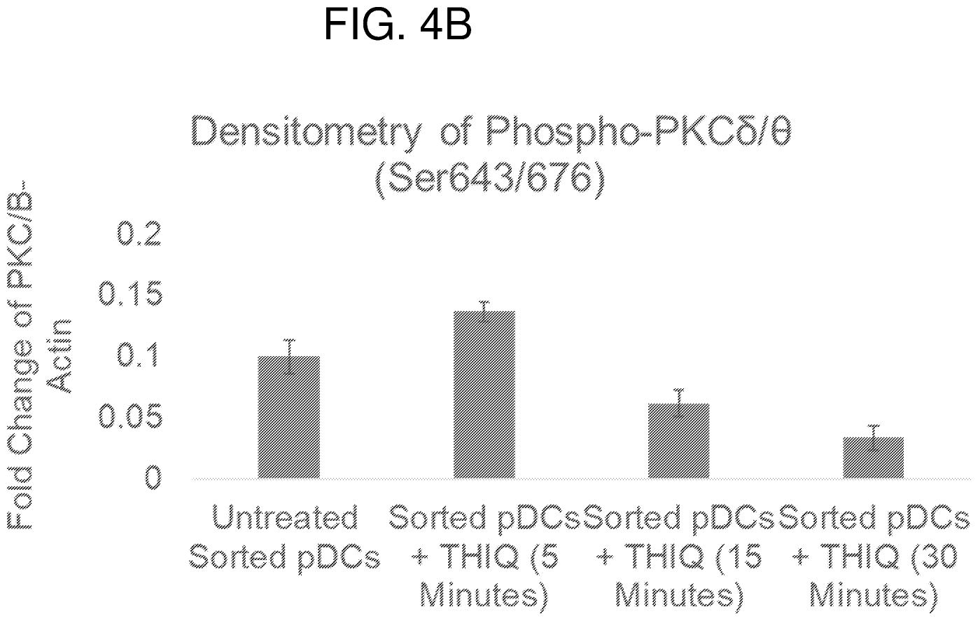

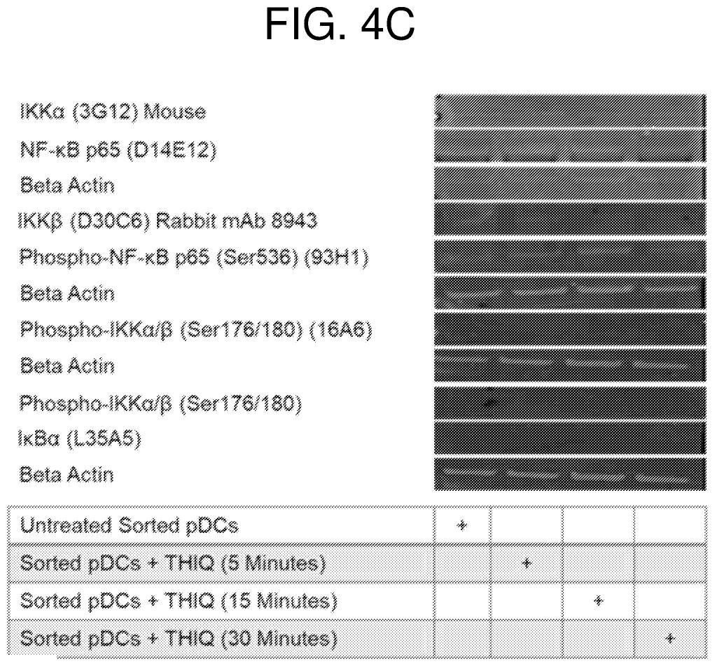

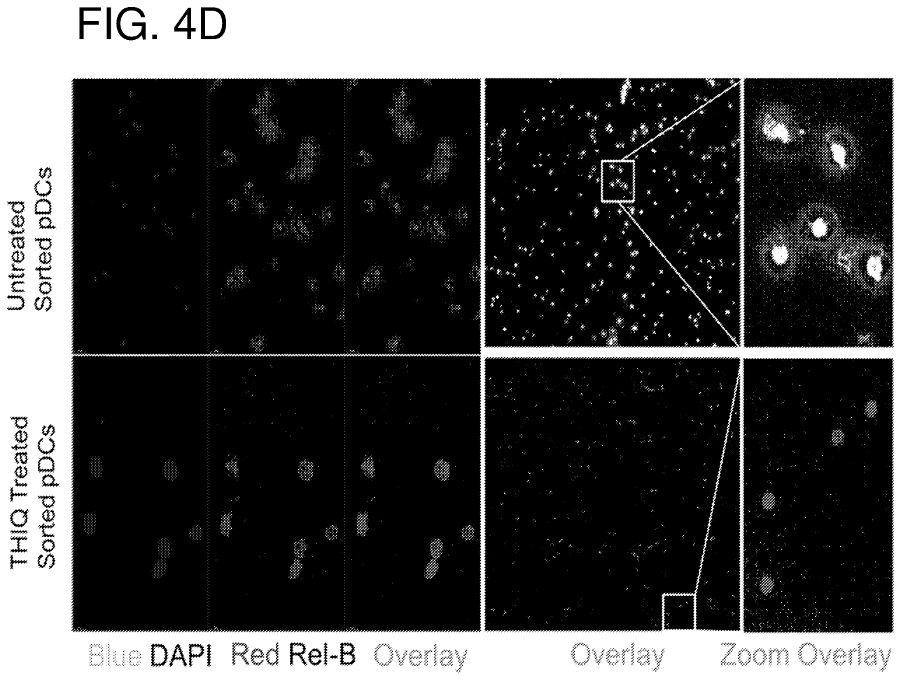

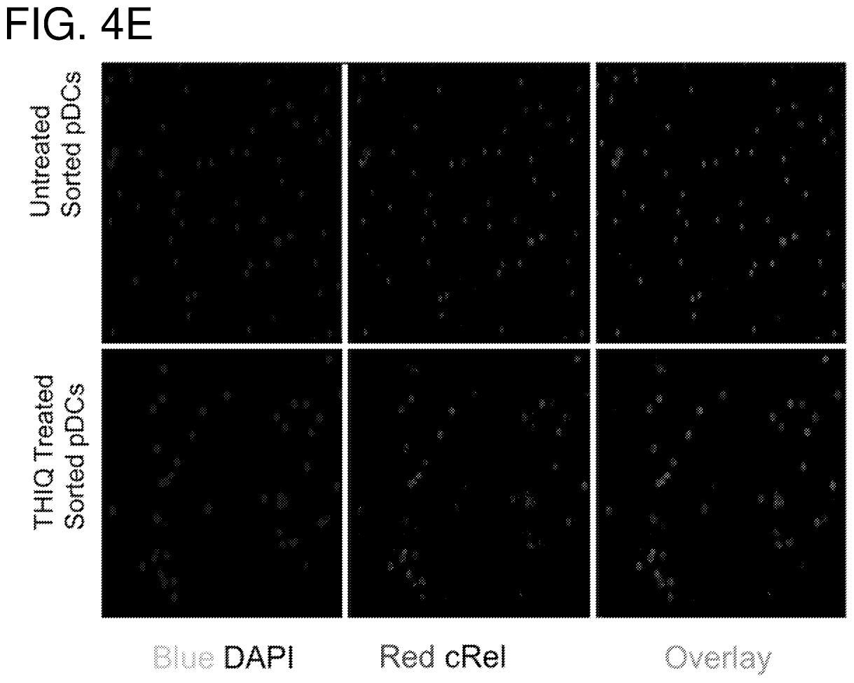

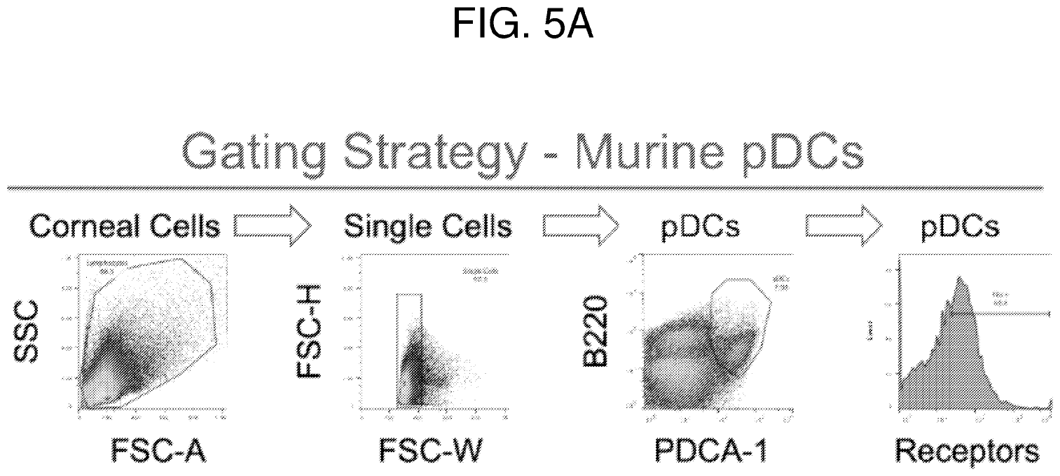

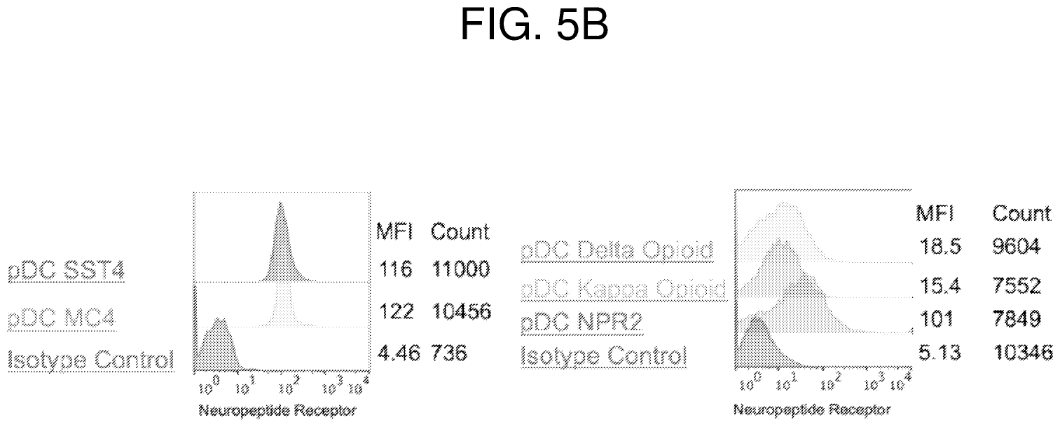

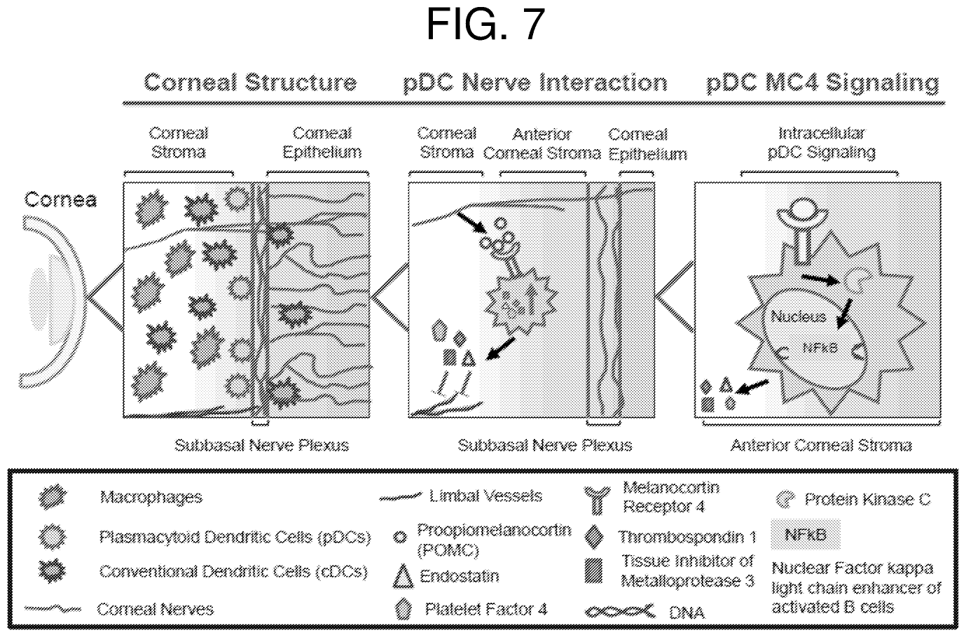

Increased phospho-PKCδ/θ (Ser643/676) phosphorylation was observed after five minutes when normalized to actin control (FIGS. 4A and 4B). PKCδ/θ (Ser643/676) phosphorylation decreased after THIQ treatment at 15 and 30 minutes when normalized to actin controls. Phosphorylation of additional PKC isoforms was not observed after THIQ treatment, however some basal expression of PKD/PKCμ was observed (FIG. 4A). Basal PKCθ (Ser676) phosphorylation has been reported in leukocytes such as primary CD4+ T cells (Wang et al., Front. Immunol. 3:197, 2012) and has been observed to increase upon TCR activation. PKCθ has been reported to activate the transcription factor nuclear factor kappa B (NF-kB) (Altman et al., Immunol. Rev. 192:53-63, 2003). NF-kB nuclear localization of pDCs treated with THIQ (10 ug/mL) for 24 hours was examined (FIG. 4C). c-Rel and Relb allow for nuclear localization of NF-kB. Increased RelB (FIG. 4D) nuclear localization but not c-Rel (FIG. 4E) was observed in THIQ treated pDCs compared to control pDCs. Taken together, these data suggest that MC4 signaling in pDCs is mediated through PKC-6 and NF-kB signaling (FIG. 7).

### Example 6. Activation of Opioid Receptors or Somatostatin Receptors on Plasmacytoid Dendritic Cells Increases Production of Angiostatic Factors

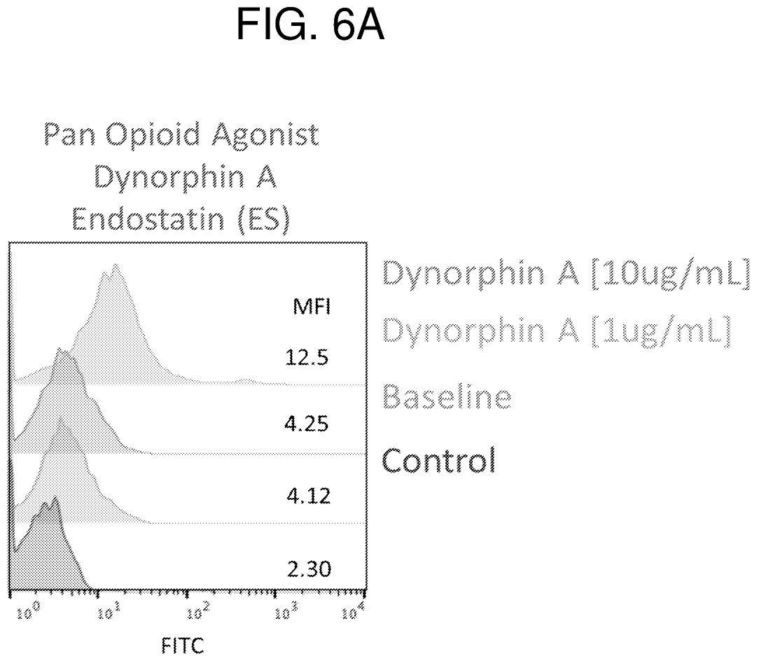

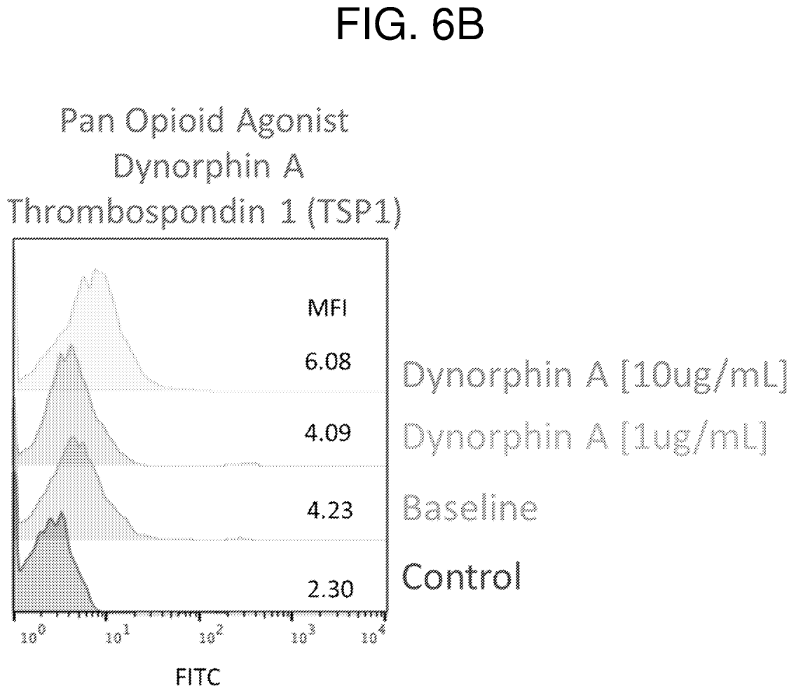

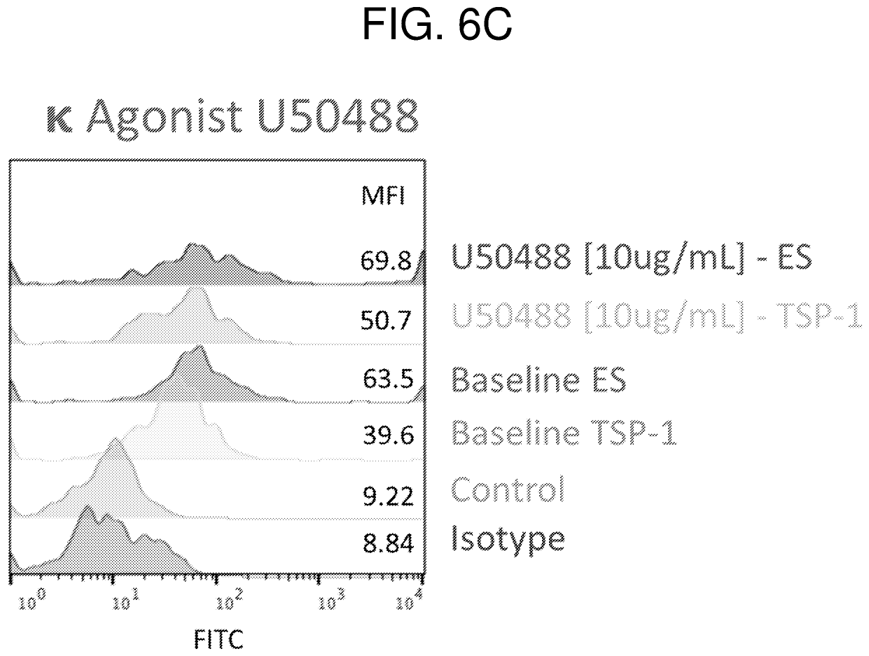

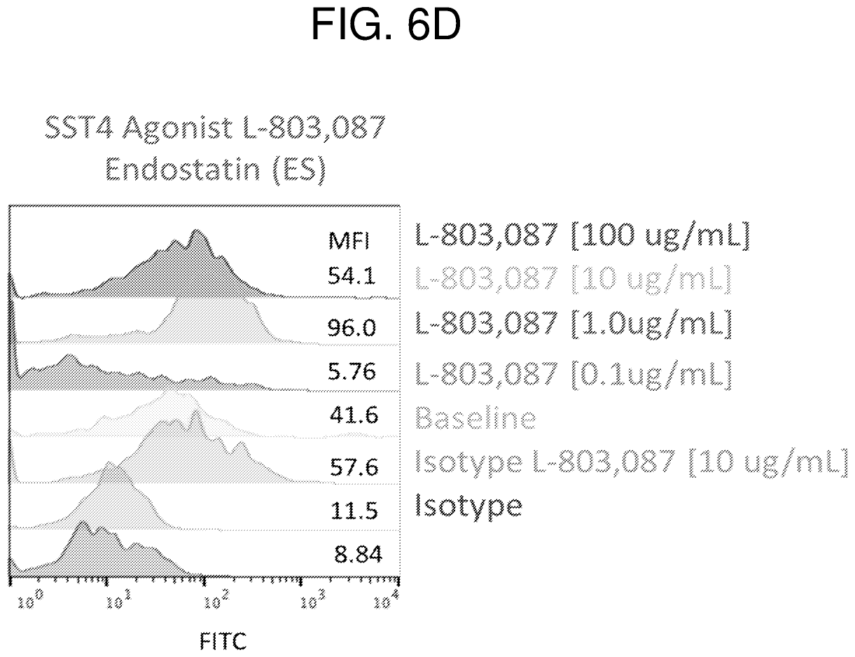

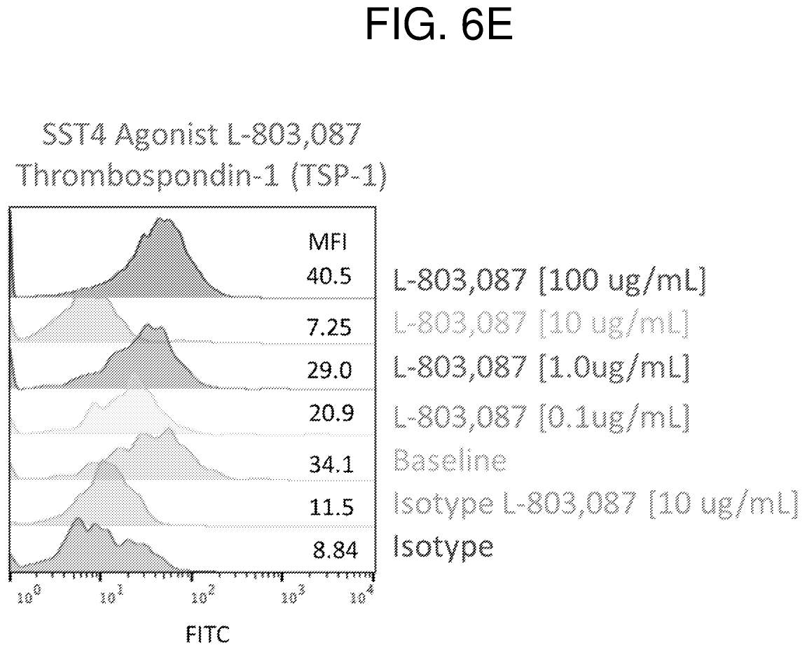

The role of pDC opioid (delta, kappa, and mu) and somatostatin receptor 4 (SST4) on pDC angiostatic activity was examined. At the outset, pan opioid receptor activation using Dynorphin A was tested. Splenocytes were isolated from three C57BL/6 mice and incubated for 24 hours with the pan opioid agonist Dynorphin A. Cells were then stained for pDC markers, PDCA-1, B220, and the angiostatic molecules ES and TSP-1. Dynorphin increased pDC ES and TSP-1 protein expression compared to baseline levels (FIGS. 6A and 6B). Previous studies have established a role for the kappa opioid receptor in the direct modulation of angiogenesis but have not examined leukocyte-expressed receptors. To further examine the role of pDC opioid receptors, the impact of the kappa receptor agonist, U50488, was examined. The kappa opioid agonist increased pDC ES and TSP-1 expression after 24 hours compared to baseline levels (FIG. 6C).

Next, the role of the SST4 receptor on pDC angiostatic molecule expression was examined. SST4 has been previously shown to modulate angiogenesis, however it has not been examined on pDCs. We found the selective SST4 agonist, L-803,087, had a mixed impact on pDC ES and TSP-1 expression compared to baseline (FIG. 6D). Combined, these findings indicate that opioid receptor agonists and SST4 agonists regulate the expression of angiostatic molecules by pDCs.

### Statistical Analysis

Results are presented as mean±standard deviation with statistical significance determined for by either two-tailed student t-test or 1-way ANOVA with a Tukey post hoc test (Prism GraphPad Software, La Jolla, Calif.) to account for multiple comparison testing. Significance was assigned based on p<0.05 for uniformity.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Some embodiments are within the scope of the following numbered paragraphs.

1. A method of treating or preventing an ocular disease or condition characterized by neovascularization and/or inflammation in a subject, the method comprising activating a neuropeptide receptor on plasmacytoid dendritic cells (pDCs) in the subject (e.g., pDCs of the eye).
2. The method of paragraph 1, wherein the neovascularization and/or inflammation is corneal neovascularization and/or inflammation.
3. The method of paragraph 1 or 2, wherein the subject has or is at risk of developing corneal infection, inflammation, autoimmune disease, limbal stem cell deficiency, neoplasia, uveitis, keratitis, corneal ulcers, glaucoma, rosacea, lupus, dry eye disease, or ocular damage due to trauma, corneal graft rejection, surgery, or contact lens wear.
4. The method of paragraph 2 or 3, wherein the disease or condition is episcleritis, scleritis, uveitis, or retinal vasculitis.
5. The method of paragraph 1, wherein the neovascularization and/or inflammation is retinal neovascularization and/or inflammation.
6. The method of paragraph 5, wherein the subject has or is at risk of developing ischemic retinopathy, diabetic retinopathy, retinopathy of prematurity, retinal vein occlusion, ocular ischemic syndrome, sickle cell disease, Eales' disease, or macular degeneration.
7. The method of paragraph 1, wherein the neovascularization and/or inflammation is choroidal neovascularization and/or inflammation.
8. The method of paragraph 7, wherein the subject has or is at risk of developing inflammatory neovascularization with uveitis, macular degeneration, ocular trauma, sickle cell disease, pseudoxanthoma elasticum, angioid streaks, optic disc drusen, myopia, malignant myopic degeneration, or histoplasmosis.
9. The method of any one of paragraphs 1-8, wherein activating a neuropeptide receptor on pDCs in the subject comprises administering a neuropeptide receptor agonist to the subject.
10. The method of paragraph 9, wherein the neuropeptide receptor is a melanocortin (MC) receptor, a somatostatin (SST) receptor, or an opioid receptor.
11. The method of paragraph 10, wherein the MC receptor is an MC4 receptor.
12. The method of paragraph 10 or 11, wherein the MC receptor is an MC1, MC2, MC3, or MC5 receptor.
13. The method of paragraph 10, wherein the SST receptor is an SST1, SST2, SST3, SST4, or SST5 receptor.
14. The method of paragraph 10, wherein the opioid receptor is a delta opioid receptor, kappa opioid receptor, or mu opioid receptor.
15. The method of any one of paragraphs 9-14, wherein the neuropeptide receptor agonist is ((3R)—N-[(2R)-3-(4-chlorophenyl)-1-[4-cyclohexyl-4-(1,2,4-triazol-1-ylmethyl)piperidin-1-yl]-1-oxopropan-2-yl]-1,2,3,4-tetrahydroisoquinoline-3-carboxamide (THIQ), PF-00446687, PL-6983, or any one of the neuropeptide receptor agonists recited in Tables 1-3.
16. The method of any one of paragraphs 1-15, wherein activating the neuropeptide receptor on pDCs in the subject increases expression of one or more angiostatic neuropeptides, increases phosphorylation of protein kinase Cδ/θ (PKCδ/θ), and/or increases nuclear localization of nuclear factor kappa B (NF-κB) in the pDCs.
17. The method of paragraph 16, wherein the one or more angiostatic neuropeptides are selected from the group consisting of endostatin (ES), platelet factor 4 (PF4), thrombospondin 1 (TSP-1), and tissue inhibitor of matrix metalloprotease three (TIMP3).
18. The method of any one of paragraphs 1-17, wherein the neuropeptide receptor agonist is administered to the eye of the subject.
19. The method of paragraph 18, wherein the neuropeptide receptor agonist is administered to the eye of the subject using intravitreal injection, sub-retinal injection, sub-conjunctival injection, sub-corneal injection, eye drops, ophthalmic pellets, drug-eluting contact lenses, ophthalmic plugs, ophthalmic depot, or intraocular device.
20. The method of any one of paragraphs 1-17, wherein the neuropeptide receptor agonist is administered to the subject by way of systemic administration.
21. The method of paragraph 20, wherein the systemic administration comprises intravenous injection.
22. The method of any one of paragraphs 1-21, wherein the pDC is in the eye of the subject.
23. The method of any one of paragraphs 1-22, wherein the subject is human.
24. A pharmaceutical composition comprising a neuropeptide receptor agonist and a pharmaceutically acceptable ophthalmic carrier or diluent.
25. A kit comprising the pharmaceutical composition of paragraph 24, a topical anesthetic eye drop, and a package insert.
26. The kit of paragraph 25, wherein the package insert instructs a user of the kit to perform the method of any one of paragraphs 1 to 23 (e.g., paragraph 18 or 19).

Other embodiments are in the claims.

What is claimed is:

1. A method of treating or preventing an ocular disease or condition characterized by neovascularization and/or inflammation in a subject, the method comprising activating a neuropeptide receptor on plasmacytoid dendritic cells (pDCs) in the subject.

2. The method of claim 1, wherein the neovascularization and/or inflammation is corneal neovascularization and/or inflammation.

3. The method of claim 1, wherein the subject has or is at risk of developing corneal infection, inflammation, autoimmune disease, limbal stem cell deficiency, neoplasia, uveitis, keratitis, corneal ulcers, glaucoma, rosacea, lupus, dry eye disease, or ocular damage due to trauma, corneal graft rejection, surgery, or contact lens wear.

4. The method of claim 2, wherein the disease or condition is episcleritis, scleritis, uveitis, or retinal vasculitis.

5. The method of claim 1, wherein the neovascularization and/or inflammation is retinal neovascularization and/or inflammation.

6. The method of claim 5, wherein the subject has or is at risk of developing ischemic retinopathy, diabetic retinopathy, retinopathy of prematurity, retinal vein occlusion, ocular ischemic syndrome, sickle cell disease, Eales' disease, or macular degeneration.

7. The method of claim 1, wherein the neovascularization and/or inflammation is choroidal neovascularization and/or inflammation.

8. The method of claim 7, wherein the subject has or is at risk of developing inflammatory neovascularization with uveitis, macular degeneration, ocular trauma, sickle cell disease, pseudoxanthoma elasticum, angioid streaks, optic disc drusen, myopia, malignant myopic degeneration, or histoplasmosis.

9. The method of claim 1, wherein activating a neuropeptide receptor on pDCs in the subject comprises administering a neuropeptide receptor agonist to the subject.

10. The method of claim 9, wherein the neuropeptide receptor is a melanocortin (MC) receptor, a somatostatin (SST) receptor, or an opioid receptor.

11. The method of claim 10, wherein the MC receptor is an MC4 receptor.

12. The method of claim 10, wherein the MC receptor is an MC1, MC2, MC3, or MC5 receptor.

13. The method of claim 10, wherein the SST receptor is an SST1, SST2, SST3, SST4, or SST5 receptor.

14. The method of claim 10, wherein the opioid receptor is a delta opioid receptor, kappa opioid receptor, or mu opioid receptor.

15. The method of claim 9, wherein the neuropeptide receptor agonist is ((3R)—N-[(2R)-3-(4-chlorophenyl)-1-[4-cyclohexyl-4-(1,2,4-triazol-1-ylmethyl)piperidin-1-yl]-1-oxopropan-2-yl]-1,2,3,4-tetrahydroisoquinoline-3-carboxamide (THIQ), PF-00446687, PL-6983, or any one of the neuropeptide receptor agonists recited in Tables 1-3.

16. The method of claim 1, wherein activating the neuropeptide receptor on pDCs in the subject increases expression of one or more angiostatic neuropeptides, increases phosphorylation of protein kinase Cδ/θ (PKCδ/θ), and/or increases nuclear localization of nuclear factor kappa B (NF-κB) in the pDCs.

17. The method of claim 16, wherein the one or more angiostatic neuropeptides are selected from the group consisting of endostatin (ES), platelet factor 4 (PF4), thrombospondin 1 (TSP-1), and tissue inhibitor of matrix metalloprotease three (TIMP3).

18. The method of claim 1, wherein the neuropeptide receptor agonist is administered to the eye of the subject.

19. The method of claim 18, wherein the neuropeptide receptor agonist is administered to the eye of the subject using intravitreal injection, sub-retinal injection, sub-conjunctival injection, sub-corneal injection, eye drops, ophthalmic pellets, drug-eluting contact lenses, ophthalmic plugs, ophthalmic depot, or intraocular device.

20. The method of claim 1, wherein the neuropeptide receptor agonist is administered to the subject by way of systemic administration.

21. The method of claim 20, wherein the systemic administration comprises intravenous injection.

22. The method of claim 1, wherein the pDC is present in the eye of the subject.

23. The method of claim 1, wherein the subject is human.

24. A pharmaceutical composition comprising a neuropeptide receptor agonist and a pharmaceutically acceptable ophthalmic carrier or diluent.

25. A kit comprising the pharmaceutical composition of claim 24, a topical anesthetic eye drop, and a package insert.

26. The kit of claim 25, wherein the package insert instructs a user of the kit to perform the method of claim 18.
